(19) United States
(12) Patent Application Publication
(10) Pub. No.: US 2010/0160349 A1
(43) Pub. Date: Jun. 24, 2010

(54) TRIAZOLOPYRIMIDINE DERIVATIVES AS GLYCOGEN SYNTHASE KINASE 3 INHIBITORS

(76) Inventors: Christopher John Love, Deurne (BE); Ludwig Paul Cooymans, Beerse (BE); Nele Vandermaesen, Olmen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/813,973

(22) PCT Filed: Jan. 13, 2006
(86) PCT No.: PCT/EP06/50206

§ 371 Date: Jul. 13, 2007

(30) Foreign Application Priority Data
Jan. 14, 2005 (EP) 05100221.0

(51) Int. Cl. A61K 31/519; C07D 487/04; A61P 7/12; A61P 25/28; A61P 25/16; A61P 25/18; A61P 25/24; A61P 37/06; A61P 17/00; A61P 3/10; A61P 35/00

(52) U.S. Cl. 514/261.1; 544/254

(57) ABSTRACT

This invention concerns a compound of formula

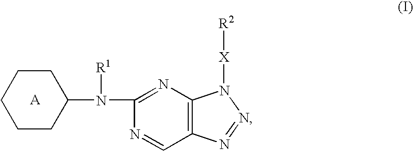

a N-oxide, a pharmaceutically acceptable addition salt, a quaternary amine and a stereochemically isomeric form thereof, wherein ring A represents phenyl, pyridyl, pyrimidinyl, pyridazinyl or pyrazinyl; R¹ represents hydrogen; aryl; formyl; C₁₋₆alkylcarbonyl; C₁₋₆alkyl; C₁₋₆alkyloxycarbonyl; C₁₋₆alkyl substituted with formyl, C₁₋₆alkylcarbonyl, C₁₋₆alkyloxycarbonyl or C₁₋₆alkylcarbonyloxy; X represents a direct bond; —(CH₂)ₙ₃— or —(CH₂)ₙ₄—X₁ₐ—X_{1b}—; R² represents an optionally substituted cyclic system; provided that N,3-diphenyl-3H-1,2,3-triazolo[4,5-d]pyrimidin-5-amine is not included; their use, pharmaceutical compositions comprising them and processes for their preparation.

The present invention concerns a novel group of compounds, their use as a medicine, their use for the manufacture of a medicament for the treatment of a disease mediated through glycogen synthase kinase 3 (GSK3), in particular glycogen synthase kinase 3α and 3β; processes for their preparation and pharmaceutical compositions comprising them.

WO 00/62778 describes cyclic protein tyrosine kinase inhibitors. In particular, it discloses thiazolyl derivatives comprising a bicyclic ring system.

WO 01/44246 describes bicyclic pyrimidine and pyridine based compounds having GSK3 inhibiting activity.

WO 99/65897 describes pyrimidine and pyridine based compounds having GSK3 inhibiting activity.

WO 02/04450 describes purine derivatives having the activity of either inhibiting the formation of amyloid beta or stimulating the formation of sbeta-amyloid precursor protein.

WO 02/50073 describes pyrazolo[3,4-c]pyridines as GSK-3 inhibitors.

WO 2004/018473 relates to di- and trisubstituted 8-aza purine derivatives as cyclin-dependent kinase inhibitors.

JP 59062594 describes 3,5-disubstituted triazolopyrimidine compounds.

The present invention relates to compounds, which are distinguishable from the prior art in structure, pharmacological activity, potency, selectivity, solubility, permeability, metabolic stability.

The present invention concerns a compound of formula (I)

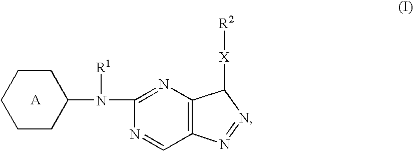

a N-oxide, a pharmaceutically acceptable addition salt, a quaternary amine and a stereochemically isomeric form thereof, wherein
ring A represents phenyl, pyridyl, pyrimidinyl, pyridazinyl or pyrazinyl;
R¹ represents hydrogen; aryl; formyl; C₁₋₆alkylcarbonyl; C₁₋₆alkyl; C₁₋₆alkyloxycarbonyl; C₁₋₆alkyl substituted with formyl, C₁₋₆alkylcarbonyl, C₁₋₆alkyloxycarbonyl or C₁₋₆alkylcarbonyloxy;
X represents a direct bond; —(CH₂)ₙ₃— or —(CH₂)ₙ₄—X₁ₐ—X_{1b}—;
   with n₃ representing an integer with value 1, 2, 3 or 4;
   with n₄ representing an integer with value 1 or 2;
   with X₁ₐ representing O, C(═O) or NR³; and
   with X_{1b} representing a direct bond or C₁₋₂alkyl;
R² represents C₃₋₇cycloalkyl; phenyl; a 4, 5, 6- or 7-membered monocyclic heterocycle containing at least one heteroatom selected from O, S or N; benzoxazolyl or a radical of formula

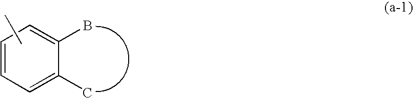

wherein —B—C— represents a bivalent radical of formula

—CH₂—CH₂—CH₂— (b-1);

—CH₂—CH₂—CH₂—CH₂— (b-2);

—X₁—CH₂—CH₂—(CH₂)ₙ— (b-3);

—X₁—CH₂—(CH₂)ₙ—X₁— (b-4);

—X₁—(CH₂)_{n′}—CH═CH— (b-5);

—CH═N—X₁— (b-6);
with X₁ representing O or NR³;
   n representing an integer with value 0, 1, 2 or 3;
   n′ representing an integer with value 0 or 1;
      wherein said R² substituent, where possible, may optionally be substituted with at least one substituent selected from halo; hydroxy; C₁₋₆alkyl optionally substituted with at least one R⁸ substituent; C₂₋₆alkenyl or C₂₋₆alkynyl, each optionally substituted with at least one R⁸ substituent; polyhaloC₁₋₆alkyl optionally substituted with at least one R⁸ substituent; C₁₋₆alkyloxy optionally substituted with at least one R⁸ substituent; polyhaloC₁₋₆alkyloxy optionally substituted with at least one R⁸ substituent; C₁₋₆alkylthio; polyhaloC₁₋₆alkylthio; C₁₋₆alkyloxycarbonyl; C₁₋₆alkylcarbonyloxy; C₁₋₆alkylcarbonyl; polyhaloC₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; —NR³—CN; aryloxy; arylthio; arylcarbonyl; arylC₁₋₄alkyl; arylC₁₋₄alkyloxy; a 5- or 6-membered monocyclic heterocycle containing at least one heteroatom selected from O, S or N and said 5- or 6-membered monocyclic heterocycle optionally being substituted with at least one substituent selected from R⁷;

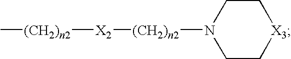

with n2 representing an integer with value 0, 1, 2, 3 or 4;
with X₂ representing O, NR³ or a direct bond;
with X₃ representing O, CH₂, CHOH, CH—N(R³)₂, NR³ or N—C(═O)—C₁₋₄alkyl;
R³ represents hydrogen; C₁₋₄alkyl or C₂₋₄alkenyl;
R⁴ and R⁵ each independently represent hydrogen; cyano; C₁₋₆alkylcarbonyl optionally substituted with C₁₋₄alkyloxy or carboxyl; C₁₋₆alkyloxycarbonyl; C₃₋₇cycloalkylcarbonyl; adamantanylcarbonyl; C₁₋₄alkyloxyC₁₋₄alkyl; C₁₋₄alkyl substituted with C₁₋₄alkyl-NR³—; C₁₋₆alkyl optionally substituted with at least one substituent selected from halo, hydroxy, cyano, carboxyl, C₁₋₄alkyloxy, polyhalo-C₁₋₄alkyl, C₁₋₄alkyloxyC₁₋₄alkyloxy, NR^{4a}R^{5a}, C(═O)NR^{4a}R^{5a} or

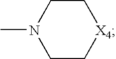

with X₄ representing O, CH₂, CHOH, CH—N(R³)₂, NR³ or N—C(═O)—C₁₋₄alkyl;
R^{4a} and R^{5a} each independently represent hydrogen; C₁₋₄alkyl; C₁₋₄alkylcarbonyl or a 5- or 6-membered monocyclic heterocycle containing at least one heteroatom selected from O, S or N;
R⁶ represents C₁₋₄alkyl optionally substituted with hydroxy; polyhaloC₁₋₄alkyl or NR⁴R⁵;
R⁷ represents halo; hydroxy; C₁₋₆alkyl optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶, C₂₋₆alkenyl or C₂₋₆alkynyl, each optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶; polyhaloC₁₋₆alkyl; C₁₋₆alkyloxy optionally substituted with carboxyl; polyhaloC₁₋₆alkyloxy; C₁₋₆alkylthio; polyhaloC₁₋₆alkylthio; C₁₋₆alkyloxycarbonyl; C₁₋₆alkylcarbonyloxy; C₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; or —NR³—CN;
R⁸ represents hydroxy, cyano, carboxyl, C₁₋₄alkyloxy, C₁₋₄alkyloxyC₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶;
n1 represents an integer with value 1 or 2;
aryl represents phenyl or phenyl substituted with at least one substituent selected from halo, C₁₋₆alkyl, C₃₋₇cycloalkyl, C₁₋₆alkyloxy, cyano, nitro, polyhaloC₁₋₆alkyl or polyhaloC₁₋₆alkyloxy,
   provided that N,3-diphenyl-3H-1,2,3-triazolo[4,5-d]pyrimidin-5-amine is not included.

The present invention also relates to the use of a compound of formula (I) for the manufacture of a medicament for the prevention or the treatment of a disease mediated through GSK3 wherein the compound of formula (I) is a compound having the following formula

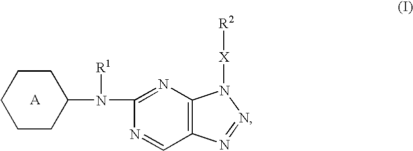

a N-oxide, a pharmaceutically acceptable addition salt, a quaternary amine and a stereochemically isomeric form thereof, wherein
ring A represents phenyl, pyridyl, pyrimidinyl, pyridazinyl or pyrazinyl;
R¹ represents hydrogen; aryl; formyl; C₁₋₆alkylcarbonyl; C₁₋₆alkyl; C₁₋₆alkyloxycarbonyl; C₁₋₆alkyl substituted with formyl, C₁₋₆alkylcarbonyl, C₁₋₆alkyloxycarbonyl or C₁₋₆alkylcarbonyloxy;
X represents a direct bond; —(CH₂)ₙ₃— or —(CH₂)ₙ₄—X₁ₐ—X_{1b}—;
   with n₃ representing an integer with value 1, 2, 3 or 4;
   with n₄ representing an integer with value 1 or 2;
   with X₁ₐ representing O, C(═O) or NR³; and
   with X_{1b} representing a direct bond or C₁₋₂alkyl;
R² represents C₃₋₇cycloalkyl; phenyl; a 4, 5, 6- or 7-membered monocyclic heterocycle containing at least one heteroatom selected from O, S or N; benzoxazolyl or a radical of formula

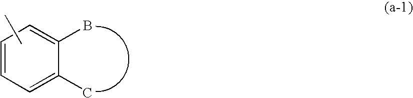

wherein —B—C— represents a bivalent radical of formula

—CH₂—CH₂—CH₂— (b-1);

—CH₂—CH₂—CH₂—CH₂— (b-2);

—X₁—CH₂—CH₂—(CH₂)ₙ— (b-3);

—X₁—CH₂—(CH₂)ₙ—X₁— (b-4);

—X₁—(CH₂)_{n′}—CH═CH— (b-5);

—CH═N—X₁— (b-6);
with X₁ representing O or NR³;
n representing an integer with value 0, 1, 2 or 3;
n′ representing an integer with value 0 or 1;
   wherein said R² substituent, where possible, may optionally be substituted with at least one substituent selected from halo; hydroxy; C₁₋₆alkyl optionally substituted with at least one R⁸ substituent; C₂₋₆alkenyl or C₂₋₆alkynyl, each optionally substituted with at least one R⁸ substituent; polyhaloC₁₋₆alkyl optionally substituted with at least one R⁸ substituent; C₁₋₆alkyloxy optionally substituted with at least one R⁸ substituent; polyhaloC₁₋₆alkyloxy optionally substituted with at least one R⁸ substituent; C₁₋₆alkylthio; polyhaloC₁₋₆alkylthio; C₁₋₆alkyloxycarbonyl; C₁₋₆alkylcarbonyloxy; C₁₋₆alkylcarbonyl; polyhaloC₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; —NR³—CN; aryloxy; arylthio; arylcarbonyl; arylC₁₋₄alkyl; arylC₁₋₄alkyloxy; a 5- or 6-membered monocyclic heterocycle containing at least one heteroatom selected from O, S or N and said 5- or 6-membered monocyclic heterocycle optionally being substituted with at least one substituent selected from R⁷; or

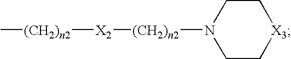

with n2 representing an integer with value 0, 1, 2, 3 or 4;
with X₂ representing O, NR³ or a direct bond;
with X₃ representing O, CH₂, CHOH, CH—N(R³)₂, NR³ or N—C(═O)—C₁₋₄alkyl;
R³ represents hydrogen; C₁₋₄alkyl or C₂₋₄alkenyl;
R⁴ and R⁵ each independently represent hydrogen; cyano; C₁₋₆alkylcarbonyl optionally substituted with C₁₋₄alkyloxy or carboxyl; C₁₋₆alkyloxycarbonyl; C₃₋₇cycloalkylcarbonyl; adamantanylcarbonyl; C₁₋₄alkyloxyC₁₋₄alkyl; substituted with C₁₋₄alkyl-NR³—; C₁₋₆alkyl optionally substituted with at least one substituent selected from halo, hydroxy, cyano, carboxyl, C₁₋₄alkyloxy, polyhalo-C₁₋₄alkyloxyC₁₋₄alkyloxy, NR^{4a}R^{5a}, C(═O)NR^{4a}R^{5a} or

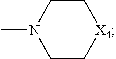

with X₄ representing O, CH₂, CHOH, CH—N(R³)₂, NR³ or N—C(═O)—C₁₋₄alkyl;
R^{4a} and R^{5a} each independently represent hydrogen; C₁₋₄alkyl; C₁₋₄alkylcarbonyl or a 5- or 6-membered monocyclic heterocycle containing at least one heteroatom selected from O, S or N;
R⁶ represents C₁₋₄alkyl optionally substituted with hydroxy; polyhaloC₁₋₄alkyl or NR⁴R⁵;
R⁷ represents halo; hydroxy; C₁₋₆alkyl optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶, C₂₋₆alkenyl or C₂₋₆alkynyl, each optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶; polyhaloC₁₋₆alkyl; C₁₋₆alkyloxy optionally substituted with carboxyl; polyhaloC₁₋₆alkyloxy; C₁₋₆alkylthio; polyhaloC₁₋₆alkylthio; C₁₋₆alkyloxycarbonyl; C₁₋₆alkylcarbonyloxy; C₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; or —NR³—CN;
R⁸ represents hydroxy, cyano, carboxyl, C₁₋₄alkyloxy, C₁₋₄alkyloxyC₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶;
n1 represents an integer with value 1 or 2;
aryl represents phenyl or phenyl substituted with at least one substituent selected from halo, C₁₋₆alkyl, C₃₋₇cycloalkyl, C₁₋₆alkyloxy, cyano, nitro, polyhaloC₁₋₆alkyl or polyhaloC₁₋₆alkyloxy.

As used herein C₁₋₂alkyl as a group or part of a group defines straight or branched chain saturated hydrocarbon radicals having from 1 to 2 carbon atoms such as methyl, ethyl; C₁₋₃alkyl as a group or part of a group defines straight or branched chain saturated hydrocarbon radicals having from 1 to 3 carbon atoms such as such as the groups defined for C₁₋₂alkyl and propyl, 1-methylethyl; C₁₋₄alkyl as a group or part of a group defines straight or branched chain saturated hydrocarbon radicals having from 1 to 4 carbon atoms such as the groups defined for C₁₋₃alkyl and butyl; C₁₋₆alkyl as a group or part of a group defines straight or branched chain saturated hydrocarbon radicals having from 1 to 6 carbon atoms such as the groups defined for C₁₋₄alkyl and pentyl, hexyl, 2-methylbutyl and the like; C₂₋₄alkenyl as a group or part of a group defines straight and branched chain hydrocarbon radicals having from 2 to 4 carbon atoms and containing a double bond such as ethenyl, propenyl, butenyl and the like; C₂₋₆alkenyl as a group or part of a group defines straight and branched chain hydrocarbon radicals having from 2 to 6 carbon atoms and containing a double bond such as the groups defined for C₂₋₄alkenyl and pentenyl, hexenyl and the like; C₂₋₆alkynyl as a group or part of a group defines straight and branched chain hydrocarbon radicals having from 2 to 6 carbon atoms and containing a triple bond such as such as ethynyl, propynyl, butynyl, pentynyl, hexynyl and the like; C₃₋₇cycloalkyl is generic to cyclopropyl, cyclobutyl, cyclopentyl, cyclohexyl and cycloheptyl; a 4, 5, 6- or 7-membered monocyclic heterocycle containing at least one heteroatom selected from O, S or N comprises saturated, partially saturated or aromatic 4, 5, 6- or 7-membered monocyclic heterocycles containing at least one heteroatom selected from O, N or S; saturated heterocycles are heterocycles containing only single bonds; partially saturated heterocycles are heterocycles containing at least one double bond provided that the ring system is not an aromatic ring system; the term aromatic is well known to a person skilled in the art and designates cyclically conjugated systems of 4n′+2 electrons, that is with 6, 10, 14 etc. π-electrons (rule of Hückel; n′ being 1, 2, 3 etc.).

Particular examples of 4, 5, 6- or 7-membered saturated monocyclic heterocycles are azetidinyl, oxetanyl, tetrahydrofuranyl, pyrrolidinyl, dioxolanyl, imidazolidinyl, thiazolidinyl, tetrahydrothienyl, dihydrooxazolyl, isothiazolidinyl, isoxazolidinyl, oxadiazolidinyl, triazolidinyl, thiadiazolidinyl, pyrazolidinyl, piperidinyl, hexahydropyrimidinyl, hexahydropyridazinyl, dioxanyl, morpholinyl, dithianyl, thiomorpholinyl, piperazinyl, homopiperidinyl (azepanyl), [1,3]diazepanyl, homopiperazinyl ([1,4]diazepanyl), [1,2]diazepanyl, oxepanyl, dioxepanyl.

Particular examples of 5- or 6-membered partially saturated heterocycles are pyrrolinyl, imidazolinyl, pyrazolinyl and the like.

Particular examples of 4, 5, 6- or 7-membered aromatic monocyclic heterocycles are pyrrolyl, furyl, thienyl, imidazolyl, oxazolyl, isoxazolyl, thiazolyl, isothiazolyl, pyrazolyl, triazolyl, thiadiazolyl, oxadiazolyl, tetrazolyl, pyridyl, pyrimidinyl, pyrazinyl, pyridazinyl.

As used herein before, the term (═O) forms a carbonyl moiety when attached to a carbon atom, a sulfoxide moiety when attached to a sulfur atom and a sulfonyl moiety when two of said terms are attached to a sulfur atom.

The term halo is generic to fluoro, chloro, bromo and iodo. As used in the foregoing and hereinafter, polyhaloC₁₋₄alkyl and polyhaloC₁₋₆alkyl as a group or part of a group are defined as mono- or polyhalosubstituted C₁₋₄alkyl or C₁₋₆alkyl, for example, methyl substituted with one or more fluoro atoms, for example, difluoromethyl or trifluoromethyl, 1,1-difluoro-ethyl and the like. In case more than one halogen atoms are attached to an alkyl group within the definition of polyhaloC₁₋₄alkyl or polyhaloC₁₋₆alkyl, they may be the same or different.

The term heterocycle as in the definition of for instance R² is meant to include all the possible isomeric forms of the heterocycles, for instance, pyrrolyl also includes 2H-pyrrolyl.

The hereinabove-mentioned heterocycles may be attached to the remainder of the molecule of formula (I) through any ring carbon or heteroatom as appropriate, if not otherwise specified. Thus, for example, when the 5- or 6-membered heterocycle is imidazolyl, it may be 1-imidazolyl, 2-imidazolyl, 4-imidazolyl and the like.

When any variable (eg. R⁴, R⁵ etc.) occurs more than one time in any constituent, each definition is independent.

Lines drawn into ring systems from substituents indicate that the bond may be attached to any of the suitable ring atoms of the ring system. For instance for a radical of formula (a-1), said radical may be attached to the remainder of the compound of formula (I) via a carbon atom of the phenyl moiety or via a carbon atom or heteroatom of the —B—C— moiety.

For therapeutic use, salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable addition salts as mentioned hereinabove are meant to comprise the therapeutically active non-toxic acid addition salt forms which the compounds of formula (I) are able to form. The latter can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds of formula (I) containing acidic protons may be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanediol, hydrabamine salts, and salts with amino acids such as, for example, arginine, lysine and the like. Conversely the salt form can be converted by treatment with acid into the free acid form.

The term addition salt also comprises the hydrates and solvent addition forms which the compounds of formula (I) are able to form. Examples of such forms are e.g. hydrates, alcoholates and the like.

The term “quaternary amine” as used hereinbefore defines the quaternary ammonium salts which the compounds of formula (I) are able to form by reaction between a basic nitrogen of a compound of formula (I) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

The N-oxide forms of the present compounds are meant to comprise the compounds of formula (I) wherein one or several tertiary nitrogen atoms are oxidized to the so-called N-oxide.

The term “stereochemically isomeric forms” as used hereinbefore defines all the possible stereoisomeric forms which the compounds of formula (I), and their N-oxides, addition salts, quaternary amines or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of formula (I) and their N-oxides, salts, solvates or quaternary amines substantially free, i.e. associated with less than 10%, preferably less than 5%, in particular less than 2% and most preferably less than 1% of the other isomers. In particular, stereogenic centers may have the R- or S-configuration; substituents on bivalent cyclic (partially) saturated radicals may have either the cis- or trans-configuration. Compounds encompassing double bonds can have an E or Z-stereochemistry at said double bond. Stereochemically isomeric forms of the compounds of formula (I) are obviously intended to be embraced within the scope of this invention.

Some of the compounds of formula (I) may also exist in their tautomeric form (e.g. keto-enol tautomerism). Such forms although not explicitly indicated in the above formula are intended to be included within the scope of the present invention.

Whenever used hereinafter, the term “compounds of formula (I)” is meant to also include their N-oxide forms, their salts, their quaternary amines and their stereochemically isomeric forms. Of special interest are those compounds of formula (I) which are stereochemically pure.

A first interesting embodiment of the present invention are those compounds of formula (I), a N-oxide, a pharmaceutically acceptable addition salt, a quaternary amine and a stereochemically isomeric form thereof, wherein
ring A represents phenyl, pyridyl, pyrimidinyl, pyridazinyl or pyrazinyl;
R¹ represents hydrogen; aryl; formyl; C₁₋₆alkylcarbonyl; C₁₋₆alkyl; C₁₋₆alkyloxycarbonyl; C₁₋₆alkyl substituted with formyl, C₁₋₆alkylcarbonyl, C₁₋₆alkyloxycarbonyl or C₁₋₆alkylcarbonyloxy;
X represents a direct bond; —(CH₂)ₙ₃— or —(CH₂)ₙ₄—X₁ₐ—X_{1b}—;
   with n₃ representing an integer with value 1, 2, 3 or 4;
   with n₄ representing an integer with value 1 or 2;
   with X₁ₐ representing O, C(═O) or NR³; and
   with X_{1b} representing a direct bond or C₁₋₂alkyl;
R² represents C₃₋₇cycloalkyl; phenyl; a 4, 5, 6- or 7-membered monocyclic heterocycle containing at least one heteroatom selected from O, S or N; benzoxazolyl or a radical of formula

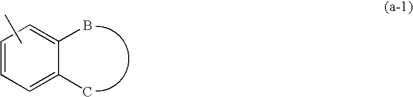

wherein —B—C— represents a bivalent radical of formula

—CH₂—CH₂—CH₂— (b-1);

—CH₂—CH₂—CH₂—CH₂— (b-2);

—X₁—CH₂—CH₂—(CH₂)ₙ— (b-3);

—X₁—CH₂—(CH₂)ₙ—X₁— (b-4);

—X₁—(CH₂)_{n′}—CH═CH— (b-5);

—CH═N—X₁— (b-6);
with X₁ representing O or NR³;
   n representing an integer with value 0, 1, 2 or 3;
   n′ representing an integer with value 0 or 1;
      wherein said R² substituent, where possible, may optionally be substituted with at least one substituent selected from halo; hydroxy; C₁₋₆alkyl optionally substituted with at least one R⁸ substituent; C₂₋₆alkenyl or C₂₋₆alkynyl, each optionally substituted with at least one R⁸ substituent; polyhaloC₁₋₆alkyl optionally substituted with at least one R⁸ substituent; C₁₋₆alkyloxy optionally substituted with at least one R⁸ substituent; polyhaloC₁₋₆alkyloxy optionally substituted with at least one R⁸ substituent; C₁₋₆alkylthio; polyhaloC₁₋₆alkylthio; C₁₋₆alkyloxycarbonyl; C₁₋₆alkylcarbonyloxy; C₁₋₆alkylcarbonyl; polyhaloC₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; —NR³—CN; aryloxy; arylthio; arylcarbonyl; arylC₁₋₄alkyl; arylC₁₋₄alkyloxy; a 5- or 6-membered monocyclic heterocycle containing at least one heteroatom selected from O, S or N and said 5- or 6-membered monocyclic heterocycle optionally being substituted with at least one substituent selected from R⁷; or

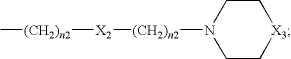

with n2 representing an integer with value 0, 1, 2, 3 or 4;
with X₂ representing O, NR³ or a direct bond;
with X₃ representing O, CH₂, CHOH, CH—N(R³)₂, NR³ or N—C(═O)—C₁₋₄alkyl;
R³ represents hydrogen; C₁₋₄alkyl or C₂₋₄alkenyl;
R⁴ and R⁵ each independently represent hydrogen; cyano; C₁₋₆alkylcarbonyl optionally substituted with C₁₋₄alkyloxy or carboxyl; C₁₋₆alkyloxycarbonyl; C₃₋₇cycloalkylcarbonyl; adamantanylcarbonyl; C₁₋₄alkyloxyC₁₋₄alkyl; C₁₋₄alkyl substituted with C₁₋₄alkyl-NR³—; C₁₋₆alkyl optionally substituted with at least one substituent selected from halo, hydroxy, cyano, carboxyl, C₁₋₄alkyloxy, polyhalo-C₁₋₄alkyl, C₁₋₄alkyloxyC₁₋₄alkyloxy, NR^{4a}R^{5a}, C(═O)NR^{4a}R^{5a} or

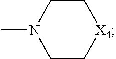

with X₄ representing O, CH₂, CHOH, CH—N(R³)₂, NR³ or N—C(═O)—C₁₋₄alkyl;
R^{4a} and R^{5a} each independently represent hydrogen; C₁₋₄alkyl; C₁₋₄alkylcarbonyl or a 5- or 6-membered monocyclic heterocycle containing at least one heteroatom selected from O, S or N;
R⁶ represents C₁₋₄alkyl optionally substituted with hydroxy; polyhaloC₁₋₄alkyl or NR⁴R⁵;
R⁷ represents halo; hydroxy; C₁₋₆alkyl optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶, C₂₋₆alkenyl or C₂₋₆alkynyl, each optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶; polyhaloC₁₋₆alkyl; C₁₋₆alkyloxy optionally substituted with carboxyl; polyhaloC₁₋₆alkyloxy; C₁₋₆alkylthio; polyhaloC₁₋₆alkylthio; C₁₋₆alkyloxycarbonyl; C₁₋₆alkylcarbonyloxy; C₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; or —NR³—CN;
R⁸ represents hydroxy, cyano, carboxyl, C₁₋₄alkyloxy, C₁₋₄alkyloxyC₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶;
n1 represents an integer with value 1 or 2;
aryl represents phenyl or phenyl substituted with at least one substituent selected from halo, C₁₋₆alkyl, C₃₋₇cycloalkyl, C₁₋₆alkyloxy, cyano, nitro, polyhaloC₁₋₆alkyl or polyhaloC₁₋₆alkyloxy;
   provided that when ring A is phenyl, X is a direct bond and R² is phenyl, then said R² phenyl must be substituted, in particular provided that when X is a direct bond and R² is phenyl, then said R² phenyl must be substituted.

A second interesting embodiment of the present invention are those compounds of formula (I) wherein
ring A represents phenyl, pyridyl, pyrimidinyl, pyridazinyl or pyrazinyl;
R¹ represents hydrogen or C₁₋₆alkyl;
X represents a direct bond; —(CH₂)ₙ₃— or —(CH₂)ₙ₄—X₁ₐ—X_{1b}—;
   with n₃ representing an integer with value 1, 2, 3 or 4;
   with n₄ representing an integer with value 1 or 2;
   with X₁ₐ representing O, C(═O) or NR³; and
   with X_{1b} representing a direct bond or C₁₋₂alkyl;
R² represents C₃₋₇cycloalkyl; phenyl; a 4, 5, 6- or 7-membered monocyclic heterocycle containing at least one heteroatom selected from O, S or N; benzoxazolyl or a radical of formula

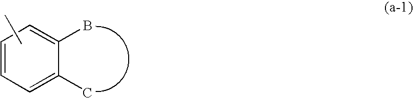

wherein —B—C— represents a bivalent radical of formula

—CH₂—CH₂—CH₂— (b-1);

—CH₂—CH₂—CH₂—CH₂— (b-2);

—X₁—CH₂—CH₂—(CH₂)ₙ— (b-3);

—X₁—CH₂—(CH₂)ₙ—X₁— (b-4);

—X₁—(CH₂)_{n′}—CH═CH— (b-5);

—CH═N—X₁— (b-6);
with X₁ representing O or NR³;
   n representing an integer with value 0, 1, 2 or 3;
   n′ representing an integer with value 0 or 1;
      wherein said R² substituent, where possible, may optionally be substituted with at least one substituent with at least one substituent, in particular 1, 2 or 3 substituents, selected from halo; C₁₋₆alkyl optionally substituted with at least one substituent selected from halo, hydroxy, cyano, carboxyl, NR⁴R⁵, C(═O)NR⁴R⁵, C₁₋₄alkyloxy or C₁₋₄alkyloxy-C₁₋₄alkyloxy; C₁₋₆alkyloxy; C₁₋₆alkyloxycarbonyl; C₁₋₄alkyloxyC₁₋₆alkyloxy; cyano; carboxyl; C(═O)NR⁴R⁵; —S(═O)ₙ₁—R⁶; arylC₁₋₄alkyloxy; or a 5- or 6-membered heterocycle containing at least one heteroatom selected from O, S or N and said 5- or 6-membered heterocycle optionally being substituted with at least one substituent selected from R⁷.

A third interesting embodiment of the present invention are the compounds of formula (I) or any subgroup thereof as mentioned hereinbefore as interesting embodiment wherein
R² represents C₃₋₇cycloalkyl; phenyl; or a 5 or 6-membered monocyclic heterocycle containing at least one heteroatom selected from O, S or N;
   wherein said R² substituent, where possible, may optionally be substituted with at least one substituent, in particular 1, 2 or 3 substituents, selected from halo; C₁₋₆alkyl optionally substituted with at least one substituent selected from halo, hydroxy, cyano, carboxyl, NR⁴R⁵, C(═O)NR⁴R⁵, C₁₋₄alkyloxy or C₁₋₄alkyloxy-C₁₋₄alkyloxy; C₁₋₆alkyloxy; C₁₋₆alkyloxycarbonyl; C₁₋₄alkyloxyC₁₋₆alkyloxy; cyano; carboxyl; C(═O)NR⁴R⁵; —S(═O)ₙ₁—R⁶; or arylC₁₋₄alkyloxy.

A fourth interesting embodiment of the present invention are the compounds of formula (I) or any subgroup thereof as mentioned hereinbefore as interesting embodiment wherein R² represents C₃₋₇cycloalkyl; phenyl; a 4, 5, 6- or 7-membered monocyclic heterocycle containing at least one heteroatom selected from O, S or N; benzoxazolyl or a radical of formula (a-1) wherein said R² substituent is substituted with at least one substituent selected from C₁₋₆alkyl substituted with NR⁴R⁵; C₂₋₆alkenyl or C₂₋₆alkynyl, each substituted with NR⁴R⁵; polyhaloC₁₋₆alkyl substituted with NR⁴R⁵; C₁₋₆alkyloxy substituted with NR⁴R⁵; polyhaloC₁₋₆alkyloxy substituted with NR⁴R⁵; or NR⁴R⁵.

A fifth interesting embodiment of the present invention are the compounds of formula (I) or any subgroup thereof as mentioned hereinbefore as interesting embodiment wherein R² represents C₃₋₇cycloalkyl or phenyl; in particular phenyl;

wherein said R² substituent, where possible, may optionally be substituted with at least one substituent, in particular 1, 2 or 3 substituents, selected from halo; C₁₋₆alkyl optionally substituted with at least one substituent selected from halo, hydroxy, cyano, carboxyl, NR⁴R⁵, C(═O)NR⁴R⁵, C₁₋₄alkyloxy or C₁₋₄alkyloxy-C₁₋₄alkyloxy; C₁₋₆alkyloxy; C₁₋₆alkyloxycarbonyl; C₁₋₄alkyloxyC₁₋₆alkyloxy; cyano; carboxyl; C(═O)NR⁴R⁵; —S(═O)ₙ₁—R⁶; or arylC₁₋₄alkyloxy.

A sixth interesting embodiment of the present invention are the compounds of formula (I) or any subgroup thereof as mentioned hereinbefore as interesting embodiment wherein R³ represents hydrogen;
R⁴ and R⁵, each independently represent hydrogen; C₁₋₆alkylcarbonyl optionally substituted with C₁₋₄alkyloxy; C₁₋₆alkyloxycarbonyl; C₃₋₇cycloalkylcarbonyl; adamantanylcarbonyl; C₁₋₆alkyl optionally substituted with at least one substituent selected from halo, hydroxy, carboxyl, C₁₋₄alkyloxy, polyhaloC₁₋₄alkyl, NR^{4a}R^{5a}, C(═O)NR^{4a}R^{5a},
R⁶ represents C₁₋₄alkyl optionally substituted with hydroxy, or NR⁴R⁵.

A seventh interesting embodiment of the present invention are the compounds of formula (I) or any subgroup thereof as mentioned hereinbefore as interesting embodiment wherein ring A represents phenyl or pyridyl, in particular phenyl.

An eighth interesting embodiment of the present invention are the compounds of formula (I) or any subgroup thereof as mentioned hereinbefore as interesting embodiment wherein X represents a direct bond.

A ninth interesting embodiment of the present invention are the compounds of formula (I) or any subgroup thereof as mentioned hereinbefore as interesting embodiment wherein R¹ represents hydrogen.

A tenth interesting embodiment of the present invention are the compounds of formula (I) or any subgroup thereof as mentioned hereinbefore as interesting embodiment wherein R² represents an optionally substituted phenyl, in particular substituted phenyl, more in particular phenyl substituted with one substituent.

An eleventh interesting embodiment of the present invention are the compounds of formula (I) or any subgroup thereof as mentioned hereinbefore as interesting embodiment wherein R² represents phenyl substituted with at least one substituent, in particular with one substituent, selected from halo; C₁₋₆alkyl optionally substituted with at least one substituent selected from hydroxy, cyano, carboxyl, C₁₋₄alkyloxy, C₁₋₄alkyloxyC₁₋₄alkyloxy or NR⁴R⁵.

A twelfth interesting embodiment of the present invention are those compounds of formula (I) or any subgroup thereof as mentioned hereinbefore as interesting embodiment wherein the R² substituent is substituted with 1 substituent and preferably said substituent is placed in meta or para position.

A thirteenth interesting embodiment of the present invention are the compounds of formula (I) or any subgroup thereof as mentioned hereinbefore as interesting embodiment wherein R⁴, and R⁵ each independently represent hydrogen; C₁₋₆alkylcarbonyl optionally substituted with C₁₋₄alkyloxy; C₁₋₆alkyloxycarbonyl; C₃₋₇cycloalkyl-carbonyl; adamantanylcarbonyl.

A fourteenth interesting embodiment of the present invention are the compounds of formula (I) or any subgroup thereof as mentioned hereinbefore as interesting embodiment wherein
ring A represents phenyl;
R¹ represents hydrogen;
X represents a direct bond;
R² represents phenyl optionally substituted with halo; C₁₋₆alkyl optionally substituted with at least one substituent selected from hydroxy, cyano, carboxyl, C₁₋₄alkyloxy, C₁₋₄alkyloxyC₁₋₄alkyloxy or NR⁴R⁵ wherein R⁴, R⁵ each independently represent hydrogen; C₁₋₆alkylcarbonyl optionally substituted with C₁₋₄alkyloxy; C₁₋₆alkyloxycarbonyl; C₃₋₇cycloalkyl-carbonyl; adamantanylcarbonyl.

Compounds of formula (I) can be prepared by reacting an intermediate of formula (II) with an intermediate of formula (III) in the presence of a suitable solvent, such as for example (CH₃)₂N—C(═O)H, dimethylsulfoxide, an alcohol, e.g. CH₃—O—CH₂—CH₂—OH, 2-propanol and the like, optionally in the presence of a suitable base, such as for example N,N-diisopropylethanamine, NaH or 2,6-dimethylpyridine. The solvent is in particular dimethylsulfoxide or CH₃—O—CH₂—CH₂—OH.

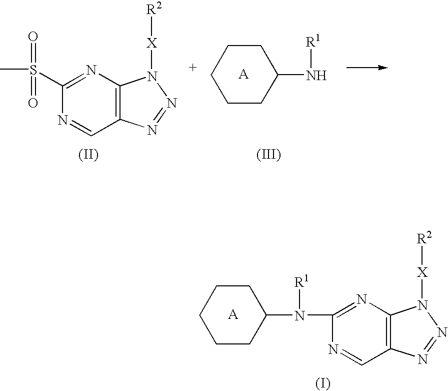

Compounds of formula (I) can also be prepared by cyclizing an intermediate of formula (IV) in the presence of a nitrite salt, such as for example NaNO₂, a suitable acid, such as for example hydrochloric acid, e.g. HCl 6N or HCl 1N, and/or acetic acid and the like, and optionally in the presence of a suitable solvent, such as for example water.

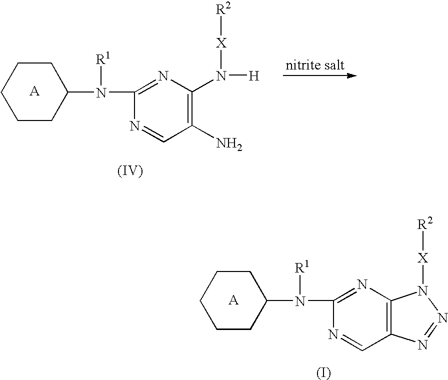

The above reaction can also be used to prepare a compound of formula (I) wherein R² represents a phenyl ring substituted with aminocarbonyl, said compound being represented by formula (I-a), from an intermediate of formula (IV) wherein R² represents a phenyl ring substituted with an imidazole moiety, said intermediate being represented by formula (IV-a).

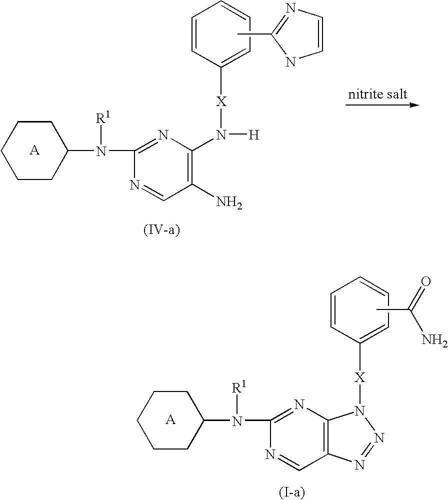

Compounds of formula (I) can also be prepared by reacting an intermediate of formula (V) with an intermediate of formula (III) in the presence of a suitable solvent, such as for example (CH₃)₂N—C(═O)H, dimethylsulfoxide, an alcohol, e.g. CH₃—O—CH₂—CH₂—OH, 2-propanol and the like, optionally in the presence of a suitable base, such as for example N,N-diisopropylethanamine, NaH or 2,6-dimethylpyridine.

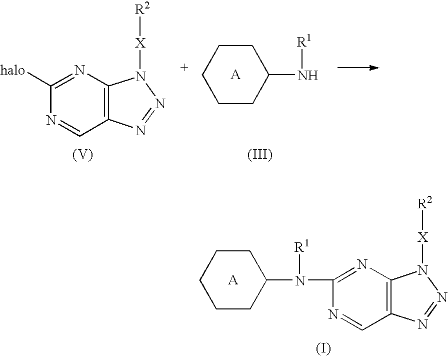

In the above reactions, the obtained compound of formula (I) can be isolated, and, if necessary, purified according to methodologies generally known in the art such as, for example, extraction, crystallization, distillation, trituration and chromatography. In case the compound of formula (I) crystallizes out, it can be isolated by filtration. Otherwise, crystallization can be caused by the addition of an appropriate solvent, such as for example water; acetonitrile; an alcohol, such as for example methanol, ethanol; and combinations of said solvents. Alternatively, the reaction mixture can also be evaporated to dryness, followed by purification of the residue by chromatography (e.g. reverse phase HPLC, flash chromatography and the like). The reaction mixture can also be purified by chromatography without previously evaporating the solvent. The compound of formula (I) can also be isolated by evaporation of the solvent followed by recrystallisation in an appropriate solvent, such as for example water; acetonitrile; an alcohol, such as for example methanol; and combinations of said solvents.

The person skilled in the art will recognise which method should be used, which solvent is the most appropriate to use or it belongs to routine experimentation to find the most suitable isolation method.

The compounds of formula (I) may further be prepared by converting compounds of formula (I) into each other according to art-known group transformation reactions.

The compounds of formula (I) may be converted to the corresponding N-oxide forms following art-known procedures for converting a trivalent nitrogen into its N-oxide form. Said N-oxidation reaction may generally be carried out by reacting the starting material of formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. t.butyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Compounds of formula (I) wherein R² is a ring system substituted with C₁₋₆alkyl substituted with NHC(═O)—O—C₁₋₆alkyl, can be converted into a compound of formula (I) wherein said R² substituent is a ring system substituted with C₁₋₆alkyl substituted with NH₂, by reaction with an acid, such as for example HCl, in the presence of a suitable solvent, such as for example dioxane and an alcohol, e.g. 2-propanol.

Compounds of formula (I) wherein R² is a ring system substituted with halo, e.g. bromo, can be converted into a compound of formula (I) wherein said R² substituent is unsubstituted, in the presence of H₂ and in the presence of a suitable catalyst, such as for example palladium on charcoal, a suitable catalyst poison, such as for example a thiophene solution, a suitable base, such as for example N,N-diethylethanamine, and a suitable solvent, such as for example tetrahydrofuran.

Compounds of formula (I) wherein R² is a ring system substituted with halo can also be converted into a compound of formula (I) wherein R² is a ring system substituted with C₁₋₆alkylthio, by reaction with a reagent of formula alkaline metal^{+ -}S—C₁₋₆alkyl, e.g. Na^{+ -}S—C₁₋₆alkyl, in the presence of a suitable solvent, such as N,N-dimethylsulfoxide. The latter compounds can further be converted into a compound of formula (I) wherein R² is a ring system substituted with C₁₋₆alkyl-S(═O)—, by reaction with a suitable oxidizing agent, such as a peroxide, e.g. 3-chlorobenzenecarboperoxoic acid, in the presence of a suitable solvent, such as an alcohol, e.g. ethanol.

Compounds of formula (I) wherein R² is a ring system which is unsubstituted, can be converted into a compound wherein R² is a ring system substituted with halo, by reaction with a suitable halogenating agent, such as, for example Br₂ or 1-(chloromethyl)-4-fluoro-1,4-diazoniabicyclo[2,2,2]octane bis[tetrafluoroborate], in the presence of a suitable solvent, such as tetrahydrofuran, water, acetonitrile, chloroform and optionally in the presence of a suitable base such as N,N-diethylethanamine.

Compounds of formula (I) wherein R² is a ring system substituted with NH₂, can be converted into a compound of formula (I) wherein R² is a ring system substituted with NH—S(═O)₂—NR⁴R⁵, by reaction with W₁-S(═O)₂—NR⁴R⁵ wherein W₁ represents a suitable leaving group such as for example a halo atom, e.g. chloro, in the presence of a suitable solvent, such as for example N,N-dimethylacetamide and a suitable base, such as for example N,N-diethylethanamine.

Compounds of formula (I) wherein R¹ is hydrogen, can be converted into a compound of formula (I) wherein R¹ is ethyl by reaction with N,N-diethylethanamine in the presence of a suitable solvent, such as for example N,N-dimethylformamide.

Compounds of formula (I) wherein R² is a ring system substituted with C(═O)—C₁₋₆alkyl, can be converted into a compound of formula (I) wherein R² is a ring system substituted with C(═O)—N(CH₃)₂, by reaction with N,N-dimethylformamide.

Some of the compounds of formula (I) and some of the intermediates in the present invention may consist of a mixture of stereochemically isomeric forms. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography, in particular liquid chromatography using a chiral stationary phase.

It is to be understood that in the above or the following preparations, the reaction products may be isolated from the reaction medium and, if necessary, further purified according to methodologies generally known in the art such as, for example, extraction, crystallization, distillation, trituration and chromatography.

Some of the intermediates and starting materials are known compounds and may be commercially available or may be prepared according to art-known procedures.

Intermediates of formula (II) can be prepared by reacting an intermediate of formula (VI) with a suitable oxidizing agent, such as for example KMnO₄, in the presence of a suitable solvent, such as for example water, and a suitable acid, such as for example acetic acid. An alternative suitable oxidizing agent is meta-chloroperbenzoic acid, in a suitable solvent, such as for example a mixture of CH₂Cl₂ and an alcohol, e.g. methanol, or CH₂Cl₂ optionally in the presence of morpholinomethyl polystyrene HL resin and (polystyrylmethyl)trimethylammonium bicarbonate resin. Another suitable oxidizing agent is an aqueous solution of H₂O₂ in the presence of a suitable acid, such as for example acetic acid.

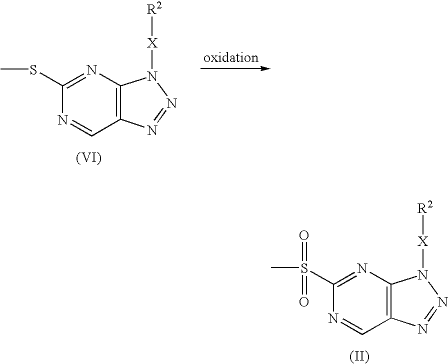

Intermediates of formula (II) wherein the ring system encompassed by R² is substituted with C₁₋₆alkyl which is substituted with NR⁴H, said R² substituent being represented by —R^{2′}—(CH₂)₁₋₆—NR⁴H and said intermediates being represented by formula (II-a), can be prepared by reacting an intermediate of formula (VI) wherein the ring system encompassed by R² is substituted with C₁₋₆alkyl which is substituted with NH₂, said R² substituent being represented by —R^{2′}—(CH₂)₁₋₆—NH₂ and said intermediate being represented by formula (VI-a), with an intermediate of formula (VII) wherein W₁ represents a suitable leaving group, such as for example halo, e.g. chloro, in the presence of a suitable oxidizing agent such as for example meta-chloroperbenzoic acid, a suitable solvent, such as for example CH₂Cl₂ and an alcohol, e.g. methanol and the like, optionally in the presence of morpholinomethyl polystyrene HL resin and (polystyrylmethyl)trimethylammonium bicarbonate resin.

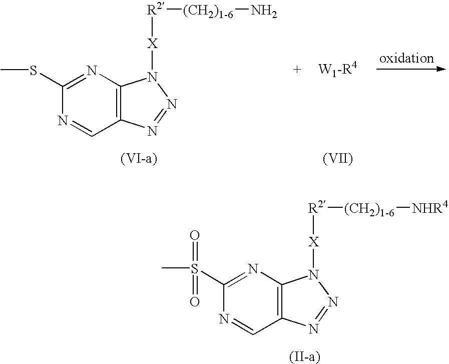

Intermediates of formula (VI) can be prepared by reacting an intermediate of formula (VIII) with a nitrite salt, such as for example NaNO₂, a suitable solvent, such as for example water, and a suitable acid, such as for example hydrochloric acid 6N or 1N optionally together with acetic acid and the like.

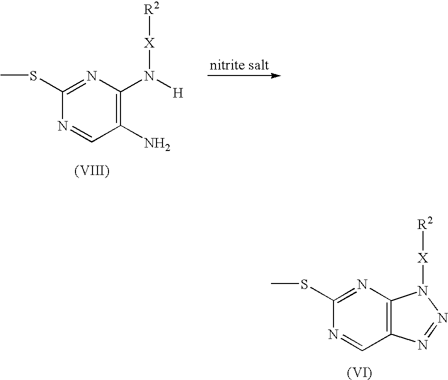

Intermediates of formula (VI-a) can be prepared by reacting an intermediate of formula (VI-b) with a suitable acid, such as for example HCl and the like, in the presence of a suitable solvent, such as for example water.

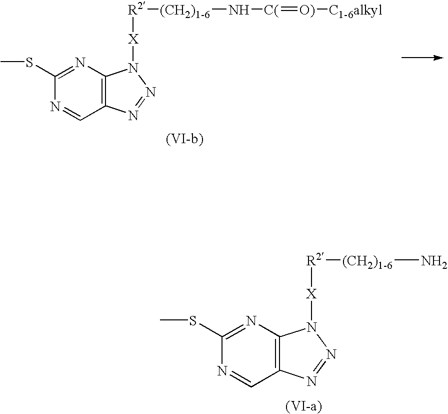

Intermediates of formula (VI-a) can be converted into an intermediate of formula (VI) wherein the ring system encompassed by R² is substituted with C₁₋₆alkyl which is substituted with NH—C(═O)—O—C₁₋₆alkyl, said R² substituent being represented by —R^{2′}—(CH₂)₁₋₆—NH—C(═O)—O—C₁₋₆alkyl and said intermediates being represented by formula (VI-c), by reaction with C₁₋₆alkyl-O—C(═O)—O—C(═O)—O—C₁₋₆alkyl in the presence of a suitable solvent, such as for example tetrahydrofuran, and a suitable base, such as for example N,N-diethylethanamine

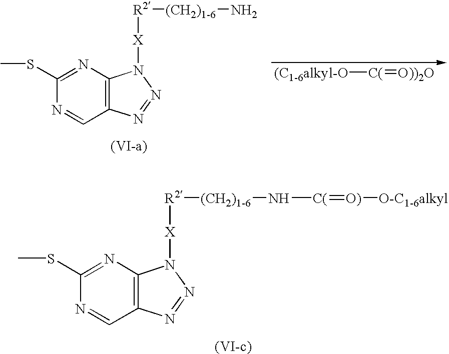

Intermediates of formula (VIII) can be prepared by reacting an intermediate of formula (IX) with a suitable reducing agent, such as for example H₂, in the presence of a suitable catalyst, such as for example platina on charcoal or palladium on charcoal, optionally a suitable catalyst poison, such as for example a thiophene solution, a suitable solvent, such as for example N,N-dimethylacetamide, tetrahydrofuran, N,N-dimethylformamide or a suitable alcohol, such as for example methanol, or mixtures thereof, and optionally in the presence of a suitable base, such as for example N,N-diethylethanamine

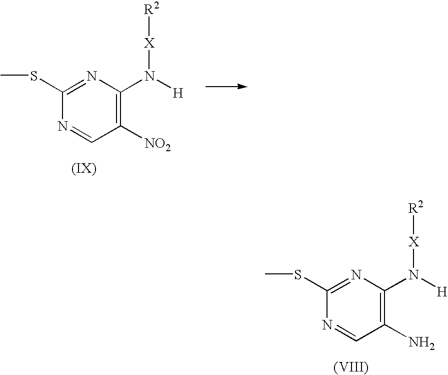

Intermediates of formula (IX) can be prepared by reacting an intermediate of formula (X), wherein W₂ represents a suitable leaving group, such as for example halo, in the presence of Na^{+ -}S—CH₃ in water.

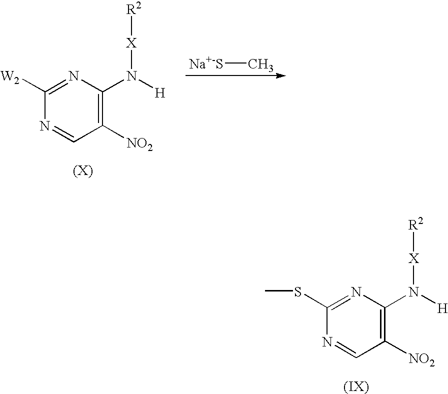

Intermediates of formula (IX) can also be prepared by reacting an intermediate of formula (XI) with an intermediate of formula (XII) wherein W₂ is as defined hereinabove, in the presence of Na^{+ -}S—CH₃ and a suitable solvent, such as for example N,N-dimethylformamide or a mixture of N,N-dimethylformamide and water, optionally in the presence of a suitable base, such as for example N,N-diisopropylethanamine

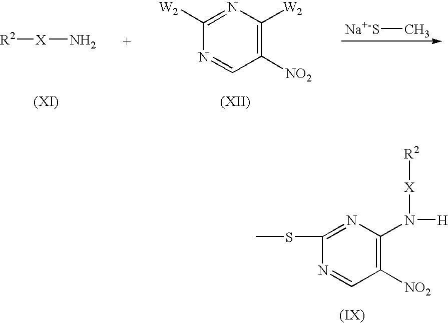

Intermediates of formula (XI) wherein X represents a direct bond, said intermediates being represented by formula (XI-a), can be prepared by reducing an intermediate of formula (XIII) in the presence of H₂, a suitable catalyst such as for example Platina on charcoal, a suitable catalyst poison, such as for example a thiophene solution, and a suitable solvent, such as for example an alcohol, e.g. methanol. Alternatively, the reduction can be performed in the presence of Fe and an ammonium chloride solution.

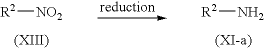

Intermediates of formula (XIII) wherein the ring system encompassed by R² is substituted with C₁₋₆alkyl which is substituted with C₁₋₄alkyloxyC₁₋₄alkyloxy, said R² substituent being represented by —R^{2′}—(CH₂)₁₋₆—O—(CH₂)₁₋₄—O—C₁₋₄alkyl and said intermediates being represented by formula (XIII-a), can be prepared by reacting an intermediate of formula (XIV) wherein W₃ represents a suitable leaving group, such as for example halo, e.g. chloro and the like, with an intermediate of formula (XV) in the presence of a suitable base, such as for example sodium hydride.

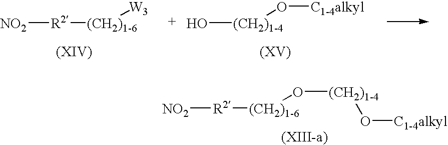

Intermediates of formula (III) wherein R¹ represents hydrogen, said intermediates being represented by formula (III-a), can be prepared by reacting an intermediate of formula (III-b) with a suitable reducing agent, such as for example H₂, in the presence of a suitable catalyst, such as for example platina on charcoal or palladium on charcoal, optionally a suitable catalyst poison, such as for example a thiophene solution, a suitable solvent, such as for example N,N-dimethylacetamide, tetrahydrofuran, N,N-dimethylformamide or a suitable alcohol, such as for example methanol, and optionally in the presence of a suitable base, such as for example N,N-diethyl-ethanamine.

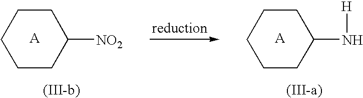

Intermediates of formula (IV) can be prepared by reducing an intermediate of formula (XVI) with a suitable reducing agent, such as for example H₂, in the presence of a suitable catalyst, such as for example platina on charcoal or palladium on charcoal, optionally in the presence of a suitable catalyst poison, such as for example a thiophene solution, optionally in the presence of NH₂—NH₂, in the presence of a suitable solvent, such as for example N,N-dimethylacetamide, tetrahydrofuran, N,N-dimethylformamide or a suitable alcohol, such as for example methanol, ethanol and the like, and optionally in the presence of a suitable base, such as for example N,N-diethylethanamine.

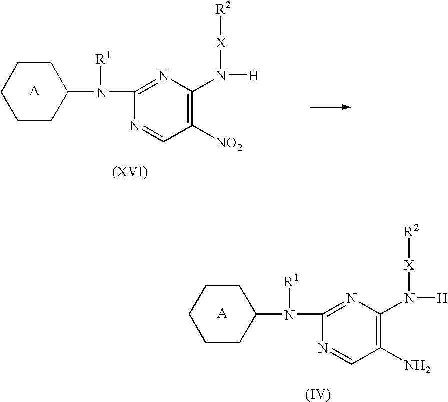

Intermediates of formula (XVI) can be prepared by reacting an intermediate of formula (XVII) wherein W₄ represents a suitable leaving group, such as for example halogen, e.g. chloro and the like, with an intermediate of formula (XI) in the presence of a suitable solvent, such as for example N,N-dimethylacetamide or an alcohol, e.g. ethanol and the like, and optionally in the presence of a suitable base, such as for example N,N-diisopropylethanamine.

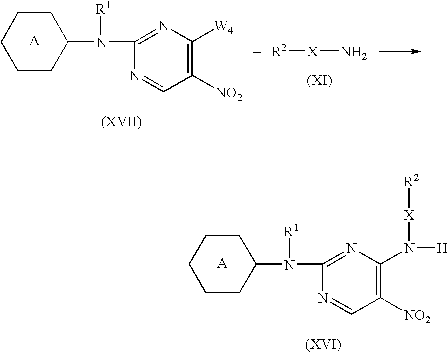

Intermediates of formula (XVI) can also be prepared by reacting an intermediate of formula (XVIII) wherein W₂ represents a suitable leaving group, such as defined above, with an intermediate of formula (III) in the presence of a suitable base, such as for example N,N-diisopropylethanamine or N,N-diethylethanamine, and optionally in the presence of a suitable solvent, such as for example N,N-dimethylacetamide, N,N-dimethylformamide, 1,4-dioxane.

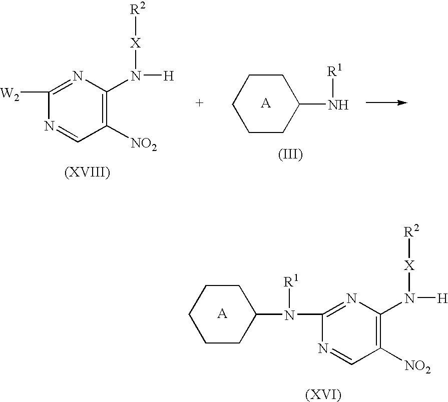

Intermediates of formula (XVI) wherein R²—X—NH— and the

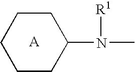

moiety represent the same substituent being represented by R^{a}—NH—, said intermediates being represented by formula (XVI-a), can be prepared by reacting an intermediate of formula (XII) wherein W₂ is defined as herein above, with R^{a}—NH₂ in the presence of a suitable base, such as for example N,N-diisopropylethanamine, and a suitable solvent, such as for example N,N-dimethyl-acetamide, N,N-dimethylformamide or CH₂Cl₂.

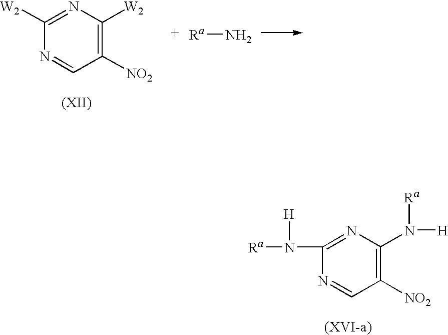

Intermediates of formula (XVI) can also be prepared by reacting an intermediate of formula (III) with an intermediate of formula (XI) and an intermediate of formula (XII) in the presence of a suitable solvent, such as for example N,N-dimethylformamide.

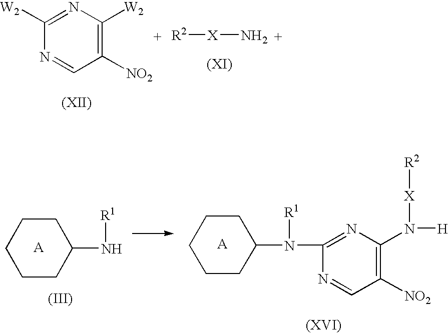

Intermediates of formula (XVII) wherein W₄ represents chloro, said intermediates being represented by formula (XVII-a), can be prepared by reacting an intermediate of formula (XIX) with POCl₃.

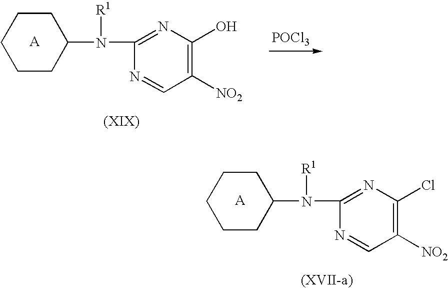

Intermediates of formula (XIX) can be prepared by reacting an intermediate of formula (III) with an intermediate of formula (XX) wherein W₅ represents a suitable leaving group, such as for example halogen, e.g. chloro, in the presence of a suitable solvent, such as for example tetrahydrofuran and water, or CH₃—O—(CH₂)₂—OH, and optionally in the presence of a suitable base, such as for example N,N-diisopropylethanamine

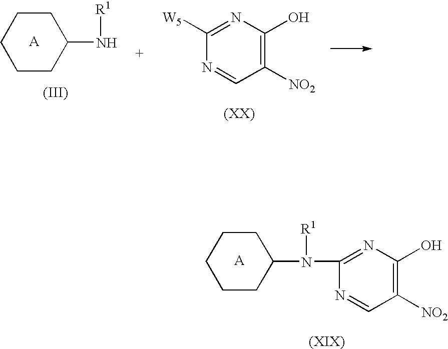

Intermediates of formula (XVIII) can be prepared by reacting an intermediate of formula (XI) with an intermediate of formula (XII) in the presence of a suitable solvent, such as for example N,N-dimethylacetamide, N,N-dimethylformamide, CH₂Cl₂ or 1,4-dioxane, and optionally in the presence of a suitable base, such as for example N,N-diisopropylethanamine

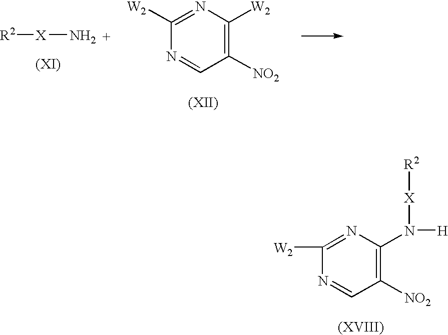

Intermediates of formula (V) can be prepared by cyclizing an intermediate of formula (XXI) in the presence of a nitrite salt, such as for example NaNO₂, a suitable acid, such as for example hydrochloric acid, e.g. HCl 6N or HCl 1N, and/or acetic acid and the like, and optionally in the presence of a suitable solvent, such as for example water.

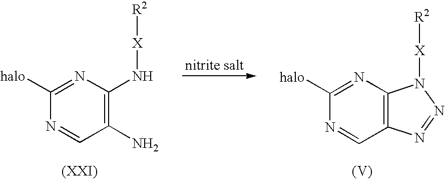

Intermediates of formula (XXI) can be prepared by reducing an intermediate of formula (XVIII) wherein W₂ represents halo, said intermediate being represented by formula (XVIII-a), with a suitable reducing agent, such as for example H₂, in the presence of a suitable catalyst, such as for example platina on charcoal, in the presence of a suitable catalyst poison, such as for example a thiophene solution, in the presence of a suitable solvent, such as for example N,N-dimethylacetamide, tetrahydrofuran, N,N-dimethyl-formamide or a suitable alcohol, such as for example methanol, ethanol and the like, and in the presence of a suitable base, such as for example N,N-diethylethanamine.

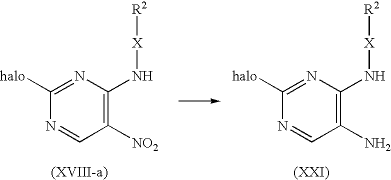

Intermediates of formula (XVIII-a) can be prepared by reacting an intermediate of formula (XI) with an intermediate of formula (XII) wherein W₂ represents halo, said intermediate being represented by formula (XII-a), in the presence of a suitable solvent, such as for example CH₂Cl₂, and a suitable base, such as for example N,N-dimethylbenzenamine.

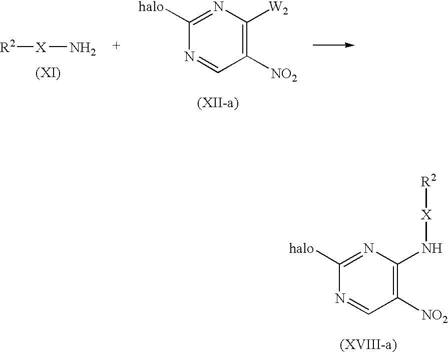

The compounds of formula (I) inhibit Glycogen synthase kinase 3 (GSK3), in particular glycogen synthase kinase 3 alpha (GSK3α) and/or glycogen synthase kinase 3 beta (GSK3β). They are selective Glycogen synthase kinase 3 inhibitors. Specific inhibitory compounds are superior therapeutic agents since they are characterized by a greater efficacy and lower toxicity by virtue of their specificity.

Synonyms for GSK3 are tau protein kinase I (TPK I), FA (Factor A) kinase, kinase FA and ATP-citrate lysase kinase (ACLK).

Glycogen synthase kinase 3 (GSK3), which exists in two isoforms as already stated above, i.e. GSK3α and GSK3β, is a proline-directed serine/threonine kinase originally identified as an enzyme that phosphorylates glycogen synthase. However, it has been demonstrated that GSK3 phosphorylates numerous proteins in vitro such as glycogen synthase, phosphatase inhibitor I-2, the type-II subunit of cAMP-dependent protein kinase, the G-subunit of phosphatase-1, ATP-citrate lyase, acetyl coenzyme A carboxylase, myelin basic protein, a microtubule-associated protein, a neurofilament protein, an N-CAM cell adhesion molecule, nerve growth factor receptor, c-Jun transcription factor, JunD transcription factor, c-Myb transcription factor, c-Myc transcription factor, L-Myc transcription factor, adenomatous polyposis coli tumor supressor protein, tau protein and β-catenin.

The above-indicated diversity of proteins which may be phosphorylated by GSK3 implies that GSK3 is implicated in numerous metabolic and regulatory processes in cells.

GSK3 inhibitors may therefore be useful in the prevention or treatment of a disease mediated through GSK3 activity such as bipolar disorder (in particular manic depression), diabetes, Alzheimer's disease, leukopenia, FTDP-17 (Fronto-temporal dementia associated with Parkinson's disease), cortico-basal degeneration, progressive supranuclear palsy, multiple system atrophy, Pick's disease, Niemann Pick's disease type C, Dementia Pugilistica, dementia with tangles only, dementia with tangles and calcification, Downs syndrome, myotonic dystrophy, Parkinsonism-dementia complex of Guam, aids related dementia, Postencephalic Parkinsonism, prion diseases with tangles, subacute sclerosing panencephalitis, frontal lobe degeneration (FLD), argyrophilic grains disease, subacute sclerotizing panencephalitis (SSPE) (late complication of viral infections in the central nervous system), inflammatory diseases, depression, cancer, dermatological disorders such as baldness, neuroprotection, schizophrenia, pain, in particular neuropathic pain. GSK3 inhibitors can also be used to inhibit sperm motility and can therefore be used as male contraceptives.

In particular, the compounds of the present invention are useful in the prevention or treatment of Alzheimer's disease; diabetes, in particular type 2 diabetes (non insulin dependent diabetes); bipolar disorder; cancer; pain, in particular neuropathic pain; depression; inflammatory diseases. More in particular, the compounds of the present invention are useful in the prevention or treatment of diabetes, in particular type 2 diabetes (non insulin dependent diabetes); pain, in particular neuropathic pain; depression; inflammatory diseases.

The major neuropathological landmarks in Alzheimer's disease are neuronal loss, the deposition of amyloid fibers and paired helical filaments (PHF) or neurofibrillary tangles (NFT). Tangle formation appears to be the consequence of accumulation of aberrantly phosphorylated tau protein. This aberrant phosphorylation destabilizes neuronal cytoskeleton, which leads to reduced axonal transport, deficient functioning and ultimately neuronal death. The density of neurofibrillary tangles has been shown to parallel duration and severity of Alzheimer's disease. Reduction of the degree of tau phosphorylation can provide for neuroprotection and can prevent or treat Alzheimer's disease or can slow the progression of the disease. As mentioned hereinabove, GSK3 phosphorylates tau protein. Thus compounds having an inhibitory activity for GSK3 may be useful for the prevention or the treatment of Alzheimer's disease.

Insulin regulates the synthesis of the storage polysaccharide glycogen. The rate-limiting step in the glycogen synthesis is catalyzed by the enzyme glycogen synthase. It is believed that glycogen synthase is inhibited by phosphorylation and that insulin stimulates glycogen synthase by causing a net decrease in the phosphorylation of this enzyme. Thus, in order to activate glycogen synthase, insulin must either activate phosphatases or inhibit kinases, or both.

It is believed that glycogen synthase is a substrate for glycogen synthase kinase 3 and that insulin inactivates GSK3 thereby promoting the dephosphorylation of glycogen synthase.

In addition to the role of GSK3 in insulin-induced glycogen synthesis, GSK3 may also play a role in insulin resistance. It is believed that GSK3 dependent Insulin Receptor Substrate-1 phosphorylation contributes to insulin resistance.

Therefore, GSK3 inhibition may result in the increased deposition of glycogen and a concomitant reduction of blood glucose, thus mimicking the hypoglycemic effect of insulin. GSK3 inhibition provides an alternative therapy to manage insulin resistance commonly observed in non insulin dependent diabetes mellitus and obesity. GSK3 inhibitors may thus provide a novel modality for the treatment of type 1 and type 2 diabetes.

GSK3 inhibitors may also be indicated for use in the prevention or the treatment of pain, in particular neuropathic pain.

After axotomy or chronic constriction injury, neuronal cells die through an apoptotic pathway and the morphological changes correlate with the onset of hyperalgesia and/or allodynia.

The induction of apoptosis is probably triggered by a reduced supply of neurotrophic factors as the time course of neuronal loss is positively altered by administration of neurotrophins. GSK has been shown to be involved in the initiation of the apoptotic cascade and trophic factor withdrawal stimulates the GSK3 apoptosis pathway. In view of the above, GSK3 inhibitors might reduce signals of and even prevent levels of neuropathic pain.

Due to their GSK3 inhibitory properties, the compounds of formula (I), their N-oxides, pharmaceutically acceptable addition salts, quaternary amines and stereochemically isomeric forms thereof, are useful to prevent or treat a GSK3 mediated disease, such as bipolar disorder (in particular manic depression), diabetes, Alzheimer's disease, leukopenia, FTDP-17 (Fronto-temporal dementia associated with Parkinson's disease), cortico-basal degeneration, progressive supranuclear palsy, multiple system atrophy, Pick's disease, Niemann Pick's disease type C, Dementia Pugilistica, dementia with tangles only, dementia with tangles and calcification, Downs syndrome, myotonic dystrophy, Parkinsonism-dementia complex of Guam, aids related dementia, Postencephalic Parkinsonism, prion diseases with tangles, subacute sclerosing panencephalitis, frontal lobe degeneration (FLD), argyrophilic grains disease, subacute sclerotizing panencephalitis (SSPE) (late complication of viral infections in the central nervous system), inflammatory diseases, depression, cancer, dermatological disorders such as baldness, neuroprotection, schizophrenia, pain, in particular neuropathic pain. The present compounds are also useful as male contraceptives. In general, the compounds of the present invention may be useful in the treatment of warm-blooded animals suffering from a disease mediated through GSK3, or they may be useful to prevent warm-blooded animals to suffer from a disease mediated through GSK3. More in particular, the compounds of the present invention may be useful in the treatment of warm-blooded animals suffering from Alzheimer's disease; diabetes, in particular type 2 diabetes; cancer; inflammatory diseases; bipolar disorder; depression; pain, in particular neuropathic pain. Even more in particular, the compounds of the present invention may be useful in the treatment of warm-blooded animals suffering from diabetes, in particular type 2 diabetes; inflammatory diseases; depression; pain, in particular neuropathic pain.

In view of the above described pharmacological properties, the compounds of formula (I) or any subgroup thereof, their N-oxides, pharmaceutically acceptable addition salts, quaternary amines and stereochemically isomeric forms, may be used as a medicine. In particular, the present compounds can be used for the manufacture of a medicament for treating or preventing a disease mediated through GSK3. More in particular, the present compounds can be used for the manufacture of a medicament for treating or preventing Alzheimer's disease; diabetes, in particular type 2 diabetes; cancer; inflammatory diseases; bipolar disorder; depression; pain, in particular neuropathic pain. Even more in particular, the present compounds can be used for the manufacture of a medicament for treating or preventing diabetes, in particular type 2 diabetes; inflammatory diseases; depression; pain, in particular neuropathic pain.

In view of the utility of the compounds of formula (I), there is provided a method of treating warm-blooded animals, including humans, suffering from or a method of preventing warm-blooded animals, including humans, to suffer from a disease mediated through GSK3, more in particular a method of treating or preventing Alzheimer's disease; diabetes, in particular type 2 diabetes; cancer; inflammatory diseases; bipolar disorder; depression; pain, in particular neuropathic pain, even more in particular diabetes, in particular type 2 diabetes; inflammatory diseases; depression; pain, in particular neuropathic pain. Said method comprises the administration, preferably oral administration, of an effective amount of a compound of formula (I), a N-oxide form, a pharmaceutically acceptable addition salt, a quaternary amine or a possible stereoisomeric form thereof, to warm-blooded animals, including humans.

The present invention also provides compositions for preventing or treating a disease mediated through GSK3, comprising a therapeutically effective amount of a compound of formula (I), a N-oxide, a pharmaceutically acceptable addition salt, a quaternary amine and a stereochemically isomeric form thereof, and a pharmaceutically acceptable carrier or diluent.

The compounds of the present invention or any subgroup thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules, and tablets.

Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment. The compounds of the present invention may also be administered via inhalation or insufflation by means of methods and formulations employed in the art for administration via this way. Thus, in general the compounds of the present invention may be administered to the lungs in the form of a solution, a suspension or a dry powder. Any system developed for the delivery of solutions, suspensions or dry powders via oral or nasal inhalation or insufflation are suitable for the administration of the present compounds.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The present compounds are orally active compounds, and are preferably orally administered.

The exact dosage, the therapeutically effective amount and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

When used as a medicament to prevent or treat Alzheimer's disease, the compounds of formula (I) may be used in combination with other conventional drugs used to combat Alzheimer's disease, such as galantamine, donepezil, rivastigmine or tacrine.

Thus, the present invention also relates to the combination of a compound of formula (I) and another agent capable of preventing or treating Alzheimer's disease. Said combination may be used as a medicine. The present invention also relates to a product containing (a) a compound of formula (I), and (b) another agent capable of preventing or treating Alzheimer's disease, as a combined preparation for simultaneous, separate or sequential use in the prevention or treatment of Alzheimer's disease. The different drugs may be combined in a single preparation together with pharmaceutically acceptable carriers.

When used as a medicament to prevent or treat type 2 diabetes, the compounds of formula (I) may be used in combination with other conventional drugs used to combat type 2 diabetes, such as glibenclamide, chlorpropamide, gliclazide, glipizide, gliquidon, tolbutamide, metformin, acarbose, miglitol, nateglinide, repaglinide, acetohexamide, glimepiride, glyburide, tolazamide, troglitazone, rosiglitazone, pioglitazone, isaglitazone.

Thus, the present invention also relates to the combination of a compound of formula (I) and another agent capable of preventing or treating type 2 diabetes. Said combination may be used as a medicine. The present invention also relates to a product containing (a) a compound of formula (I), and (b) another agent capable of preventing or treating type 2 diabetes, as a combined preparation for simultaneous, separate or sequential use in the prevention or treatment of type 2 diabetes. The different drugs may be combined in a single preparation together with pharmaceutically acceptable carriers.

When used as a medicament to prevent or treat cancer, the compounds of formula (I) may be used in combination with other conventional drugs used to combat cancer such as platinum coordination compounds for example cisplatin or carboplatin; taxane compounds for example paclitaxel or docetaxel; camptothecin compounds for example irinotecan or topotecan; anti-tumour vinca alkaloids for example vinblastine, vincristine or vinorelbine; anti-tumour nucleoside derivatives for example 5-fluorouracil, gemcitabine or capecitabine; nitrogen mustard or nitrosourea alkylating agents for example cyclophosphamide, chlorambucil, carmustine or lomustine; anti-tumour anthracycline derivatives for example daunorubicin, doxorubicin or idarubicin; HER2 antibodies for example trastzumab; and anti-tumour podophyllotoxin derivatives for example etoposide or teniposide; and antiestrogen agents including estrogen receptor antagonists or selective estrogen receptor modulators preferably tamoxifen, or alternatively toremifene, droloxifene, faslodex and raloxifene; aromatase inhibitors such as exemestane, anastrozole, letrazole and vorozole; differentiating agents for example retinoids, vitamin D and DNA methyl transferase inhibitors for example azacytidine; kinase inhibitors for example flavoperidol and imatinib mesylate or farnesyltransferase inhibitors for example R115777.

Thus, the present invention also relates to the combination of a compound of formula (I) and another agent capable of preventing or treating cancer. Said combination may be used as a medicine. The present invention also relates to a product containing (a) a compound of formula (I), and (b) another agent capable of preventing or treating cancer, as a combined preparation for simultaneous, separate or sequential use in the prevention or treatment of cancer. The different drugs may be combined in a single preparation together with pharmaceutically acceptable carriers.

When used as a medicament to prevent or treat bipolar disorder, the compounds of formula (I) may be used in combination with other conventional drugs used to combat bipolar disorder such as neuroleptica, atypical antipsychotics, anti-epileptica, benzodiazepines, lithium salts, for example olanzapine, risperidone, carbamazepine, valproate, topiramate.

Thus, the present invention also relates to the combination of a compound of formula (I) and another agent capable of preventing or treating bipolar disorder. Said combination may be used as a medicine. The present invention also relates to a product containing (a) a compound of formula (I), and (b) another agent capable of preventing or treating bipolar disorder, as a combined preparation for simultaneous, separate or sequential use in the prevention or treatment of bipolar disorder. The different drugs may be combined in a single preparation together with pharmaceutically acceptable carriers.

When used as a medicament to prevent or treat inflammatory diseases, the compounds of formula (I) may be used in combination with other conventional drugs used to combat inflammatory diseases such as steroids, cyclooxygenase-2 inhibitors, non-steroidal-anti-inflammatory drugs, TNF-α antibodies, such as for example acetyl salicylic acid, bufexamac, diclofenac potassium, sulindac, diclofenac sodium, ketorolac trometamol, tolmetine, ibuprofen, naproxen, naproxen sodium, tiaprofen acid, flurbiprofen, mefenamic acid, nifluminic acid, meclofenamate, indomethacin, proglumetacine, ketoprofen, nabumetone, paracetamol, piroxicam, tenoxicam, nimesulide, fenylbutazon, tramadol, beclomethasone dipropionate, betamethasone, beclamethasone, budesonide, fluticasone, mometasone, dexamethasone, hydrocortisone, methylprednisolone, prednisolone, prednisone, triamcinolone, celecoxib, rofecoxib, infliximab, leflunomide, etanercept, CPH 82, methotrexate, sulfasalazine.

Thus, the present invention also relates to the combination of a compound of formula (I) and another agent capable of preventing or treating inflammatory diseases. Said combination may be used as a medicine. The present invention also relates to a product containing (a) a compound of formula (I), and (b) another agent capable of preventing or treating inflammatory diseases, as a combined preparation for simultaneous, separate or sequential use in the prevention or treatment of inflammatory disorders. The different drugs may be combined in a single preparation together with pharmaceutically acceptable carriers.

When used as a medicament to prevent or treat depression, the compounds of formula (I) may be used in combination with other conventional drugs used to combat depression such as norepinephrine reuptake inhibitors, selective serotonin reuptake inhibitors (SSRI's), monoamine oxidase inhibitors (MAOI's), reversible inhibitors of monoamine oxidase (RIMA's), serotonin and noradrenaline reuptake inhibitors (SNRI's), noradrenergic and specific serotonergic antidepressants (NaSSA's), corticotropin releasing factor (CRF) antagonists, α-adrenoreceptor antagonists and atypical antidepressants.

Suitable examples of norepinephrine reuptake inhibitors include amitriptyline, clomipramine, doxepin, imipramine, trimipramine, amoxapine, desipramine, maprotiline, nortriptyline, protriptyline, reboxetine and pharmaceutically acceptable salts thereof.

Suitable examples of selective serotonin reuptake inhibitors include fluoxetine, fluvoxamine, paroxetine, sertraline and pharmaceutically acceptable salts thereof.

Suitable examples of monoamine oxidase inhibitors include isocarboxazid, phenelzine, tranylcypromine, selegiline and pharmaceutically acceptable salts thereof.

Suitable examples of reversible inhibitors of monoamine oxidase include moclobemide and pharmaceutically acceptable salts thereof.

Suitable examples of serotonin and noradrenaline reuptake inhibitors include venlafaxine and pharmaceutically acceptable salts thereof.

Suitable atypical antidepressants include bupropion, lithium, nefazodone, trazodone, viloxazine, sibutramine and pharmaceutically acceptable salts thereof.

Other suitable antidepressants include adinazolam, alaproclate, amineptine, amitriptyline/chlordiazepoxide combination, atipamezole, azamianserin, bazinaprine, befuraline, bifemelane, binodaline, bipenamol, brofaromine, bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, monirelin, nebracetam, nefopam, nialamide, nomifensine, norfluoxetine, orotirelin, oxaflozane, pinazepam, pirlindone, pizotyline, ritanserin, rolipram, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine and zometapine and pharmaceutically acceptable salts thereof, and St. John's wort herb, or Hypericum perforatum, or extracts thereof.

Thus, the present invention also relates to the combination of a compound of formula (I) and another agent capable of preventing or treating depression. Said combination may be used as a medicine. The present invention also relates to a product containing (a) a compound of formula (I), and (b) another agent capable of preventing or treating depression, as a combined preparation for simultaneous, separate or sequential use in the prevention or treatment of depression. The different drugs may be combined in a single preparation together with pharmaceutically acceptable carriers.

When used as a medicament to prevent or treat pain, the compounds of formula (I) may be used in combination with other conventional drugs used to combat pain such as nonsteroidal anti-inflammatory drugs (NSAIDS), centrally acting analgesics.

Suitable nonsteroidal anti-inflammatory drugs include salicylates, such as for example acetylsalicylic acid, ethenzamide, salicylamide; para-aminophenol derivatives, such as for example paracetamol, propacetamol, phenidine; anthranilates, such as for example etofenamate, flufenamic acid, meclofenamic acid, mefenamic acid, niflumic acid; arylacetic acids, such as for example acemetacin, bufexamac, diclofenac, indomethacin, lonazolac, sulindac, tolmetin, nabumetone; arylpropionic acids, such as for example flurbiprofen, ibuprofen, ketoprofen, naproxen, tiaprofenic acid; pyrazolinone derivatives, such as for example metamizol, propyphenazone; pyrazolidine-3,5-diones, such as for example kebuzone, mofebutazone, oxyphenbutazone, phenylbutazone; arylsulfonamides, such as for example isoxicam, lornoxicam, piroxicam, tenoxicam; ketorolac; oxaprozine; Cox-2 inhibitors, such as for example celecoxib, etodolac, meloxicam, nimesulfide, rofecoxib.

Suitable centrally acting analgesics include opioid agonists, such as for example morphine and morphinane derivatives, e.g. morphine, codeine, ethylmorphine, diacetylmorphine, dihydrocodeine, etorphine, hydrocodone, hydromorphone, levorphanol, oxycodone, oxymorphone; such as for example piperidine derivatives, e.g. pethidine, ketobemidone, fentanyl, alfentanil, remifentanil, sufentanil; such as for example methadone and congeners, e.g. levomethadone, levomethadone acetate, dextromoramide, dextropropoxyphene, diphenoxylate, loperamide, piritramide; tilidine; tramadol; viminol.

Suitable centrally acting analgesics include mixed opioid agonist-antagonists and partial agonists, such as for example buprenorphine, butorphanol, dezocine, meptazinol, nalbuphine, nalorphine, pentazocine; opioid antagonists, such as for example levallorphan, naloxone, naltrexone; non-opioid compounds, such as for example carbamazepine, clonidine, flupirtine, nefopam.

Thus, the present invention also relates to the combination of a compound of formula (I) and another agent capable of preventing or treating pain. Said combination may be used as a medicine. The present invention also relates to a product containing (a) a compound of formula (I), and (b) another agent capable of preventing or treating pain, as a combined preparation for simultaneous, separate or sequential use in the prevention or treatment of bipolar disorder. The different drugs may be combined in a single preparation together with pharmaceutically acceptable carriers.

The following examples illustrate the present invention.

### EXPERIMENTAL PART

Hereinafter, “DMF” is defined as N,N-dimethylformamide, “DIPE” is defined as diisopropylether, “DMSO” is defined as dimethylsulfoxide, “THF” is defined as tetrahydrofuran, and “DMA” is defined as N,N-dimethylacetamide.

### A. Preparation of the Intermediate Compounds

### Example A1

### a. Preparation of Intermediate 1

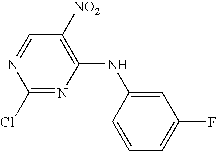

A mixture of 2,4-dichloro-5-nitropyrimidine (0.05 mol) in DMA (400 ml) was cooled to −20° C. and N-ethyl-N-(1-methylethyl)-2-propanamine (0.05 mol) was added, then a mixture of 3-fluoro-benzeneamine (0.05 mol) in DMA (200 ml) was added dropwise at −20° C. and the reaction mixture was stirred at −20° C. for 2 hours. The reaction mixture containing intermediate 1 was used as such in the next reaction step.

### b. Preparation of Intermediate 2

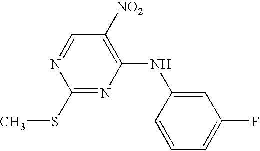

NaSCH₃, 21% in H₂O (0.05 mol) was added dropwise to intermediate 1 (0.05 mol) and the reaction mixture was stirred for 1.5 hours at room temperature, then the mixture was carefully poured out into H₂O. The resulting precipitate was stirred over the weekend, filtered off, washed and dried (vacuum). The product was crystallised from CH₃CN, then the resulting precipitate was filtered off, washed and dried (vacuum), yielding intermediate 2.

### c. Preparation of Intermediate 3

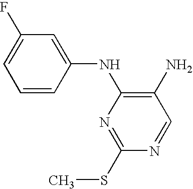

A mixture of intermediate 2 (prepared according to A1.b) (0.07 mol) and Et₃N (10 g) in THF (250 ml) was hydrogenated with Pd/C, 10% (5 g) as a catalyst in the presence of a solution of thiophene in DIPE (4% v/v, 5 ml). After uptake of H₂ (3 equiv), the catalyst was filtered off and the filtrate was evaporated. The residue was stirred in DIPE with a small amount of CH₃CN. The precipitate was filtered off and dried. Yield: 12.3 g of intermediate 3 (70.2%). The filtrate was acidified with HCl/2-propanol while stirring. The mixture was stirred for 30 minutes. The resulting precipitate was filtered off and dried. Yield: 5.17 g of intermediate 3 (25.7%).

### d. Preparation of Intermediate 4

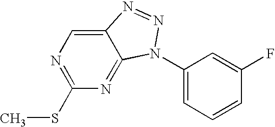

Intermediate 3 (0.08 mol) was dissolved in a mixture of 6N HCl (400 ml) and HOAc, p.a. (400 ml) and the whole was cooled to 0-5° C. A solution of NaNO₂ (0.1 mol) in H₂O (40 ml) was added dropwise over a 30 minutes period. Then, the reaction mixture was stirred for another 30 minutes while cooling on the ice-bath. Then, the mixture was stirred overnight at room temperature. The resulting precipitate was filtered off, rinsed with water, with 2-propanone, then with DIPE, and dried. Yield: 18.14 g of intermediate 4 (87%).

### e. Preparation of Intermediate 5

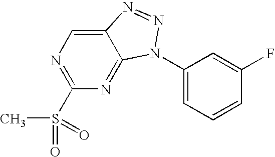

Intermediate 4 (15 g, 0.058 mol) was stirred in HOAc (700 ml) and cooled on an ice-bath. A solution of KMnO₄, p.a. (24 g, 0.15 mol) in demineralized H₂O (300 ml) was added dropwise over a 60 minutes period while cooling on an ice-bath. The mixture was stirred for one hour on the ice-bath, then for 2 hours at room temperature. Sodium bisulfite was added until a colour change resulted. EtOAc was added while stirring vigorously for a while. The mixture was stood overnight. The mixture was concentrated to ±50 ml volume. The aqueous concentrate was stirred for a while and the resulting precipitate was filtered off and dried. Yield: 11.023 g of intermediate 5 (64.8%).

### Example A2

### a. Preparation of Intermediate 6

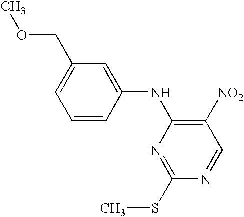

A solution of 2,4-dichloro-5-nitropyrimidine (0.047 mol) in DMF (100 ml) was cooled to −50° C. and a mixture of 3-(methoxymethyl)benzenamine (0.047 mol) in DMF (50 ml) was added dropwise, then the mixture was stirred at −50° C. for 4 hours and NaSCH₃ (0.1 mol) was added dropwise. The reaction mixture was stirred over the weekend at room temperature and the resulting precipitate was filtered off, washed with H₂O and dried (vacuum), yielding intermediate 6.

### b. Preparation of Intermediate 7

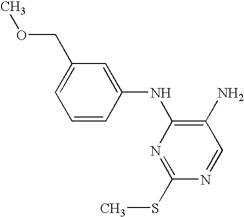

A mixture of intermediate 6 (prepared according to A2.a) (0.029 mol) in methanol (150 ml) and THF (100 ml) was hydrogenated with Pd/C (2 g) as a catalyst in the presence of thiophene solutions (4% v/v in DIPE)(2 ml). After uptake of H₂ (3 equiv., 2181 ml), the catalyst was filtered off and the filtrate was evaporated. Yield: 9 g of intermediate 7.

### c. Preparation of Intermediate 8

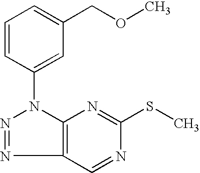

Intermediate 7 (prepared according to A2.b) (0.029 mol) was stirred in acetic acid, p.a. (100 ml) at room temperature and 1N HCl, p.a. (30 ml) was added, then a mixture of NaNO₂ (0.03 mol) in H₂O (20 ml) was added dropwise and the reaction mixture was stirred at room temperature for 1 hour. H₂O (200 ml) and a saturated NaCl solution (50 ml) were added and the mixture was extracted 3 times with EtOAc. The organic layer was evaporated and the concentrate was purified over silica gel (eluent gradient: CH₂Cl₂/Hexane from 50/50 to 100/0). The product fractions were collected and the solvent was evaporated. Yield: 5 g intermediate 8 (60%).

### d. Preparation of Intermediate 9

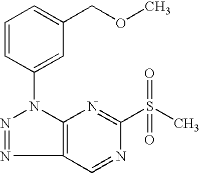

A mixture of intermediate 8 (prepared according to A2.c) (0.017 mol) in CH₂Cl₂ (200 ml) was stirred and 3-chlorobenzenecarboperoxoic acid (0.04 mol) was added at room temperature, then the reaction mixture was stirred at room temperature and washed with a calculated NaHCO₃/H₂O-solution. The organic layer was dried (MgSO₄), filtered off and the solvent was evaporated. The residue was crystallised from CH₃CN and the resulting precipitate was filtered off and dried. Yield: 3.04 g (56%) of intermediate 9. The filtrate was evaporated and the residue was crystallised from H₂O/CH₃OH. The precipitate was filtered off and dried. Yield: 1.086 g of intermediate 9 (20%).

The following intermediates were prepared according to A2.d:

| | |
|---|---|
| 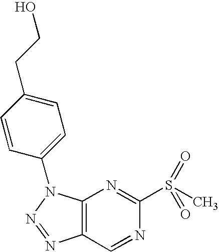 | 2-[4-(5-methanesulfonyl- [1,2,3]triazolo[4,5-d]pyrimidin-3- yl)-phenyl]-ethanol |
| 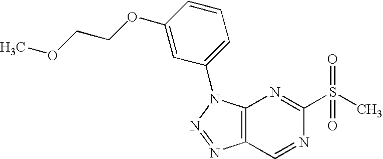 | 5-methanesulfonyl-3-[3-(2-methoxy- ethoxy)-phenyl]-3H-[1,2,3]triazolo [4,5-d]pyrimidine |

### Example A3

### a. Preparation of Intermediate 10

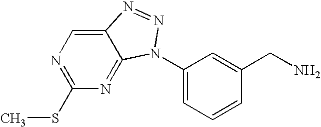

A mixture of intermediate

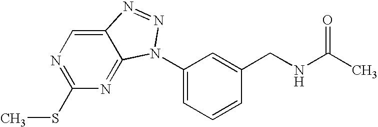

(prepared according to A2.b) (0.020 mol) in 12 N HCl, p.a. (100 ml) and H₂O (demineralised) (200 ml) was stirred and refluxed for 6 hours, then the reaction mixture was stirred over the weekend at room temperature. The resulting precipitate was filtered off and dried. Yield: 3.61 g of intermediate 10 (58.5%, m.p.: >260° C.).

Intermediate

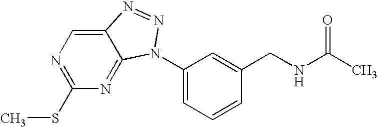

was also further reacted according to A2.d to yield

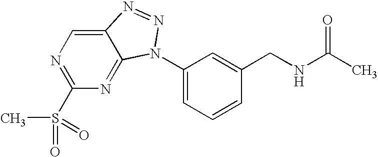

### b. Preparation of Intermediate 11

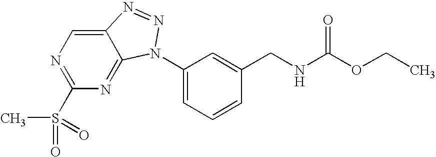

A mixture of intermediate 10 (0.005 mol) in CH₂Cl₂ (50 ml) was stirred at room temperature and morpholinomethyl Polystyrene HL resin (4 mmol/g) (0.020 mol; Novabiochem) was added, then a mixture of carbonochloridic acid ethyl ester (0.006 mol) in CH₂Cl₂ (20 ml) was added dropwise at room temperature and the reaction mixture was stirred over the weekend at room temperature. The mixture was filtered over a glass filter and the scavenger was rinsed with CH₂Cl₂/CH₃OH (30 ml; 80/20). 3-Chlorobenzenecarboperoxoic acid (0.015 mol; 70%) was added to the filtrate and the resulting mixture was stirred overnight. Extra 3-chlorobenzenecarboperoxoic acid (1 g) was added and the mixture was stirred for another 8 hours, then PS-ammonium bicarbonate scavenger (0.045 mol; Novabiochem, 3.7 mmol/g) was added and the reaction mixture was stirred overnight at room temperature. The scavenger was filtered off and the filtrate was evaporated, yielding intermediate 11.

The following intermediates were prepared according to A3.b:

| | |
|---|---|
| 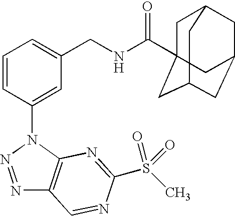 | Adamantane-1-carboxylic acid 3-(5-methane sulfonyl-[1,2,3]triazolo[4,5-d]pyrimidin-3-yl)- benzylamide |
| 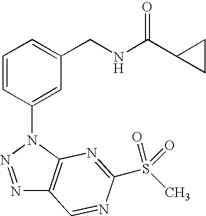 | Cyclopropanecarboxylic acid 3-(5-methane sulfonyl-[1,2,3]triazolo[4,5-d]pyrimidin-3-yl)- benzylamide |
| 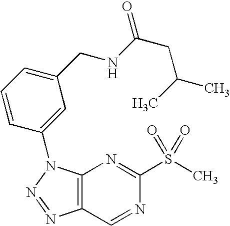 | N-[3-(5-methanesulfonyl-[1,2,3]triazolo [4,5-d]pyrimidin-3-yl)-benzyl]-3-methyl- butyramide |

### Example A4

### a. Preparation of Intermediate 12

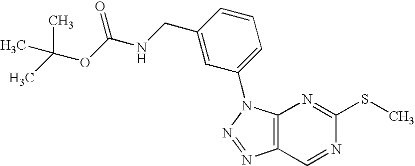

A mixture of intermediate 10 (prepared according to A3.a) (0.003 mol) and Et₃N (0.0039 mol) in THF, p.a. (20 ml) was stirred at room temperature and bis(1,1-dimethylethyl)dicarbonic acid ester (0.0033 mol) was added dropwise, then the reaction mixture was stirred overnight at room temperature and the solvent was evaporated. The residue was stirred in H₂O and dried. Yield: 1.100 g of intermediate 12 (100%).

### b. Preparation of Intermediate 13

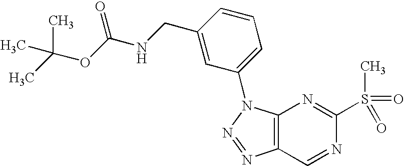

A mixture of intermediate 12 (prepared according to A4.a) (0.003 mol) in CH₂Cl₂ (50 ml) and CH₃OH (5 ml) was stirred at room temperature and then 3-chlorobenzenecarboperoxoic acid (0.006 mol, 70%) was added portionwise. The reaction mixture was stirred for 16 hours at room temperature and was washed with an equimolar aqueous NaHCO₃ solution. The organic layer was separated, dried (MgSO₄), filtered off and the solvent was evaporated. Yield: 1.157 g of intermediate 13 (95%).

### Example A5

### a. Preparation of Intermediate 14

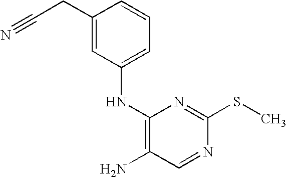

A mixture of

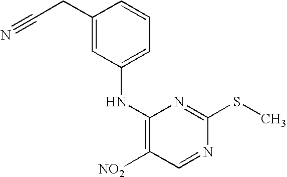

(prepared according to A2.a) (0.036 mol) in Et₃N (10 ml) and THF (250 ml) was hydrogenated with Pt/C₅% (2 g) as a catalyst in the presence of thiophene solution (4% v/v in DIPE) (1 ml). After uptake of H₂ (3 equiv.), the catalyst was filtered off and the filtrate was evaporated. The residue was taken up in a minimal amount of 2-propanone/CH₃OH (9/1) and the resulting mixture was acidified with HCl/2-propanol. The mixture was stirred over the weekend and filtered, to give a filtrate and a filter residue. The filter residue was dried. Yield: 8.00 g of intermediate 14 (72%).

### b. Preparation of Intermediate 15

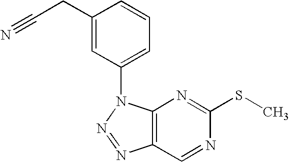

A mixture of intermediate 14 (prepared according to A5.a) (0.028 mol) in acetic acid, p.a. (60 ml) and 1N HCl, p.a. (20 ml) was stirred at room temperature and then a mixture of nitrous acid, sodium salt (0.030 mol) in H₂O (demineralised) (20 ml) was added dropwise. The reaction mixture was stirred overnight at room temperature and was diluted with ice-water (40 ml), then filtered. The filter residue was dried, yielding intermediate 15.

### c. Preparation of Intermediate 16

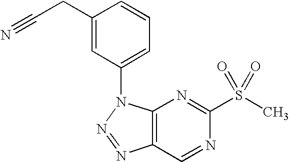

A mixture of intermediate 15 (prepared according to A5.b) (0.010 mol) in CH₂Cl₂ (80 ml) and CH₃OH (20 ml) was stirred at room temperature and 3-chlorobenzenecarboperoxoic acid (0.024 mol) was added portionwise. The reaction mixture was stirred for 3 hours at room temperature, then a mixture of NaHCO₃ (0.025 mol) in H₂O was added and the resulting mixture was stirred firmly. When the generation of gas was stopped, the layers were separated. The organic layer was dried (MgSO₄), filtered off and the solvent was evaporated. The residue was stirred in DIPE with a small amount of CH₃CN, then the precipitate was filtered off and dried. Yield: 1.218 g of intermediate 16 (39%).

The following intermediate was prepared according to A5.c:

| | |
|---|---|
| 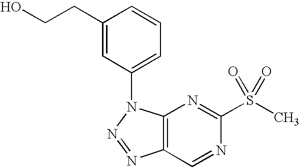 | 2-[3-(5-methanesulfonyl-[1,2,3]triazolo[4,5- d]pyrimidin-3-yl)-phenyl]-ethanol |

### Example A6

### a. Preparation of Intermediate 17

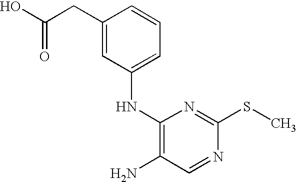

A mixture of intermediate

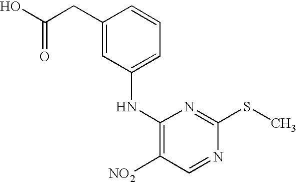

(prepared according to A2.a) (0.056 mol) in THF (150 ml) and CH₃OH (150 ml) was hydrogenated with Pd/C 10% (3 g) as a catalyst. After uptake of H₂ (3 equivalents), the catalyst was filtered off and the filtrate was evaporated. The residue was suspended from CH₃CN/H₂O and then the desired product was filtered off, washed and dried (vacuum). Yield: 4.3 g of intermediate 17.

### b. Preparation of Intermediate 18

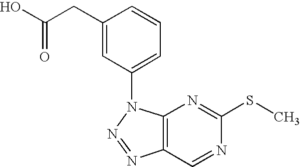

A mixture of NaNO₂ (0.018 mol) in H₂O (17 ml) was added dropwise to a solution of intermediate 17 (prepared according to A6.a) (0.015 mol) and 1N HCl (0.015 mol) in acetic acid (115 ml) and then the reaction mixture was stirred for a few hours at room temperature. The resulting precipitate was filtered off, washed and dried (vacuum). Yield: 3.6 g of intermediate 18.

### c. Preparation of Intermediate 19

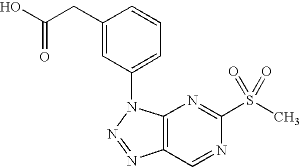

Intermediate 18 (prepared according to A6.b) (0.0017 mol) was dissolved in acetic acid (35 ml) by heating and H₂O₂ (30% in H₂O) (0.0044 mol) was added dropwise, then the reaction mixture was stirred overnight at 60° C. and the solvent was evaporated. The obtained residue was suspended in DIPE and a small amount of CH₃CN, then the resulting precipitate was filtered off, washed and dried (vacuum). Yield: 0.350 g of intermediate 19.

The following intermediate was prepared according to A6.c:

| | |
|---|---|
| 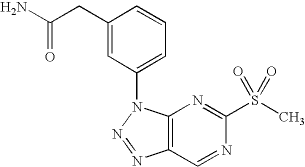 | 2-[3-(5-methanesulfonyl-[1,2,3]triazolo[4,5-d] pyrimidin-3-yl)-phenyl]-acetamide |

### Example A7

### a. Preparation of Intermediate 20

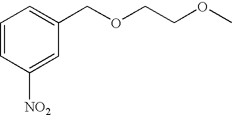

Reaction under N₂: NaH (0.07 mol) was added portionwise to 2-methoxyethanol (200 ml) cooled on an ice bath, then a solution of 1-(chloromethyl)-3-nitrobenzene (0.058 mol) in 2-methoxyethanol (q.s.) was added dropwise and the reaction mixture was stirred overnight at room temperature. The resulting precipitate was filtered off and the filtrate was evaporated, yielding intermediate 20.

### b. Preparation of Intermediate 21

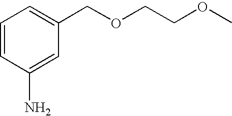

A mixture of intermediate 20 (prepared according to A7.a) (0.050 mol) in CH₃OH (150 ml) was hydrogenated with Pt/C₅% (2 g) as a catalyst in the presence of thiophene solution (4% v/v in DIPE) (1 ml). After uptake of H₂ (3 equivalents), the catalyst was filtered off and the filtrate was evaporated, yielding intermediate 21 (used as such in the next reaction step).

### c. Preparation of Intermediate 22

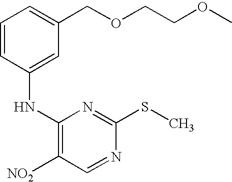

A solution of 2,4-dichloro-5-nitropyrimidine (0.026 mol) in DMF (70 ml) was cooled to −50° C. and then a mixture of intermediate 21 (prepared according to A7.b) (0.026 mol) in DMF (10 ml) was added dropwise. The resulting mixture was stirred for 2 hours at −40 a −30° C. and was then cooled to −50° C. NaSCH₃ (0.052 mol) was added dropwise and the reaction mixture was stirred overnight at room temperature. H₂O and CH₃CN were added, then the resulting precipitate was filtered off, washed and dried (vacuum). Yield: 7 g of intermediate 22.

### d. Preparation of Intermediate 23

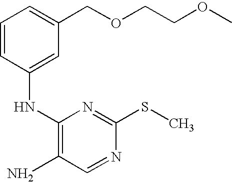

A mixture of intermediate 22 (prepared according to A7.c) (0.02 mol) in CH₃OH (250 ml) was hydrogenated at 50° C. with Pd/C 10% (2 g) as a catalyst. After uptake of H₂ (3 equivalents), the catalyst was filtered off and the filtrate was evaporated. The residue was purified by column chromatography on a glass filter (eluent gradient: CH₂Cl₂/CH₃OH 99/1->98/2). The product fractions were collected and the solvent was evaporated, yielding intermediate 23 (used as such in the next reaction step).

### e. Preparation of Intermediate 24

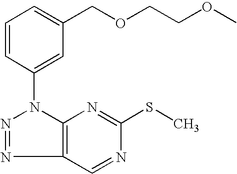

1N HCl (0.016 mol) was added to a solution of intermediate 23 (prepared according to A7.d) (0.016 mol) in acetic acid (160 ml) and then a mixture of NaNO₂ (0.018 mol) in H₂O (18 ml) was added dropwise. The reaction mixture was stirred overnight at room temperature and the solvent was evaporated. The residue was purified by column chromatography on a glass filter (eluent: CH₂Cl₂). The product fractions were collected and the solvent was evaporated, yielding intermediate 24 (used as such in the next reaction step).

### f. Preparation of Intermediate 25

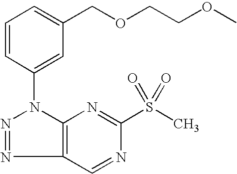

A mixture of intermediate 24 (prepared according to A7.e) (0.011 mol) and 3-chlorobenzenecarboperoxoic acid (0.022 mol) in CH₂Cl₂ (100 ml) was stirred overnight at room temperature and then extra 3-chlorobenzenecarboperoxoic acid (0.022 mol) was added. The reaction mixture was stirred for 4 hours at room temperature and a solution of NaHCO₃ in H₂O was added. The organic layer was separated, dried (MgSO₄), filtered off and the solvent was evaporated. The residue was purified by column chromatography on a glass filter (eluent: CH₂Cl₂/CH₃OH). The product fractions were collected and the solvent was evaporated. The residue was suspended in DIPE, then the desired product was filtered off, washed and dried (vacuum). Yield: 3 g of intermediate 25.

### B. Preparation of the Final Compounds

### Example B1

### a. Preparation of Compound 1

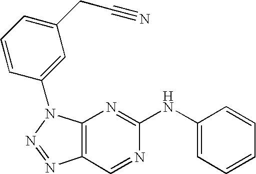

A mixture of intermediate 16 (prepared according to A5.c) (0.0002 mol) and benzenamine (0.0002 mol; 99%) in 2-methoxyethanol (1 ml) was stirred for 2 hours at 80° C. and then the reaction mixture was allowed to crystallise overnight. The resulting precipitate was filtered off and dried. Yield: 0.053 g of compound 1 (81%; melting point: 218-222° C.).

### b. Preparation of Compound 2

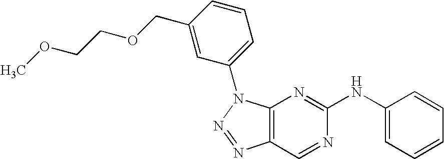

A mixture of intermediate 25 (prepared according to A7.f) (0.00028 mol) and benzenamine (0.00055 mol) in 2-methoxyethanol (2 ml) was stirred overnight at 100° C. and then H₂O and CH₃CN were added. After crystallisation, the resulting precipitate was filtered off and dried (vacuum). Yield: 0.0664 g of compound 2 (melting point: 143° C.).

### c. Preparation of Compound 3

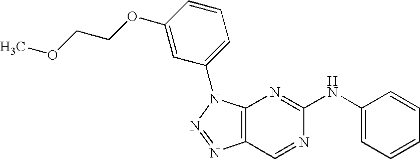

A mixture of 5-methanesulfonyl-3-[3-(2-methoxy-ethoxy)-phenyl]-3H-[1,2,3]triazolo[4,5-d]pyrimidine (prepared according to A2.d) (0.0002 mol) and benzenamine (0.0004 mol) in 2-methoxyethanol (2 ml) was stirred for 3 hours at 100° C. and the reaction mixture was stirred overnight at room temperature. H₂O and a small amount of CH₃CN were added and the mixture was heated until complete dissolution and then cooled to room temperature. The resulting precipitate was filtered off, washed and dried (vacuum), yielding compound 3 (melting point: 164° C.).

### d. Preparation of Compound 4

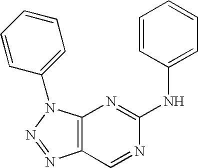

A mixture of 5-(methylsulfonyl)-3-phenyl-3H-1,2,3-triazolo[4,5-d]pyrimidine (0.0005 mol) and benzenamine (0.001 mol, p.a.) in 2-methoxyethanol (3 ml, p.a.) was stirred for 2 hours at 100° C. and the reaction mixture was diluted with EtOH (3 ml). The resulting mixture was allowed to crystallise under stirring, then the resulting precipitate was filtered off and dried. Yield: 0.100 g of compound 4 (69%, melting point: 194-198° C.)

### e. Preparation of Compound 5

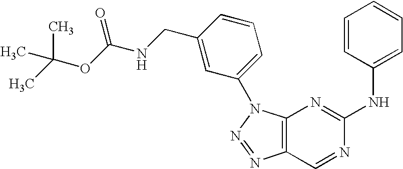

A mixture of intermediate 13 (prepared according to A4.b) (0.0003 mol) and benzenamine (0.0009 mol; 99%) in 2-methoxyethanol (3 ml) was stirred for 16 hours at 80° C., then EtOH (2 ml) was added and the reaction mixture was stirred at room temperature. The resulting precipitate was filtered off and dried. Yield: 0.082 g of compound 5 (65%, melting point: 196-198° C.).

### f. Preparation of Compound 6

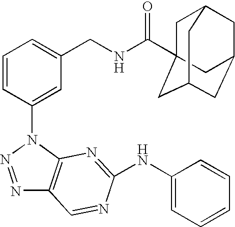

A mixture of adamantane-1-carboxylic acid 3-(5-methanesulfonyl-[1,2,3]triazolo[4,5-d]pyrimidin-3-yl)-benzylamide (prepared according to A3.b) (0.0002 mol) and benzenamine (0.0004 mol) in 2-methoxyethanol (2 ml) was stirred for 48 hours at 120° C. and then the crude mixture was purified by high-performance liquid chromatography. The product fractions were collected and the solvent was evaporated. The obtained residue was dissolved in EtOH and then the solvent was evaporated, yielding compound 6.

### g. Preparation of Compound 7

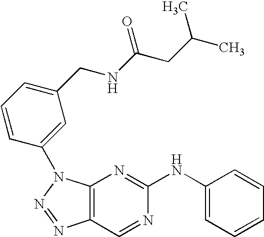

A mixture of N-[3-(5-methanesulfonyl-[1,2,3]triazolo[4,5-d]pyrimidin-3-yl)-benzyl]-3-methyl-butyramide (prepared according to A3.b) (0.0002 mol), benzenamine (0.0004 mol) in 2-methoxyethanol (q.s.) and then the crude mixture was purified by high-performance liquid chromatography. The product fractions were collected and the solvent was evaporated (Genevac). The obtained residue was dissolved in CH₃OH and then the solvent was evaporated (Genevac), yielding compound 7.

### Example B2

### Preparation of Compound 8

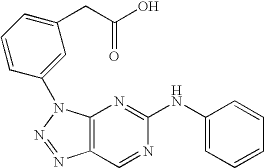

A mixture of intermediate 19 (prepared according to A6.c) (0.00024 mol) and benzenamine (0.00024 mol) in DMSO (1.5 ml) was stirred for 3 hours at 100° C., then the reaction mixture was cooled and H₂O and CH₃CN were added. After crystallisation, the formed precipitate was filtered off, washed and dried (vacuum). Yield: 0.024 g of compound 8 (melting point: 241° C.).

### Example B3

### Preparation of Compound 9

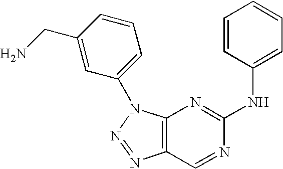

A mixture of compound 5 (prepared according to B1.e) (0.00015 mol) in 2-propanol/HCl (6M) (1 ml) and dioxane/HCl (4M) (3 ml) was stirred for 20 hours at room temperature, then the resulting precipitate was filtered off and dried.

Yield: 0.055 g of compound 9 (94%, melting point >260° C.).

Table 1 lists the compounds that were prepared according to one of the above Examples (Ex.).

TABLE 1

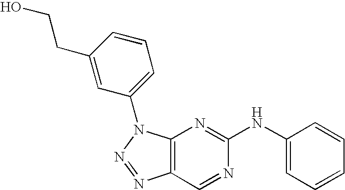

Co. No. 10; Ex. B1b; mp. 144° C.

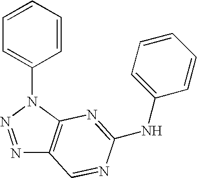

Co. No. 4; Ex. B1d; mp. 194° C.

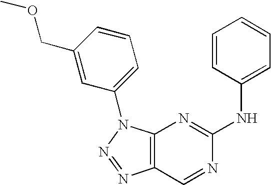

Co. No. 11; Ex. B1b; mp. 148° C.

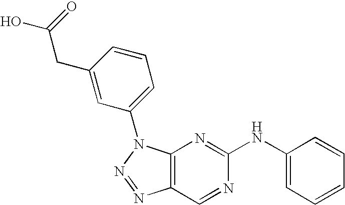

Co. No. 8; Ex. B2; mp. 241° C.

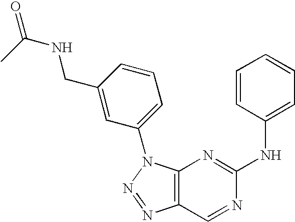

Co. No. 12; Ex. B1a; mp. 230° C.

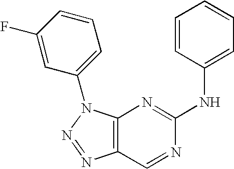

Co. No. 13; Ex. B1a; mp. 180° C.

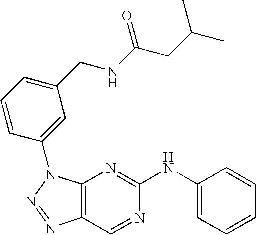

Co. No. 7; Ex. B1g;

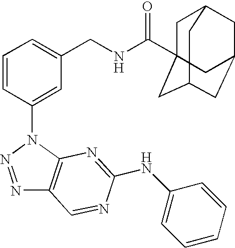

Co. No. 6; Ex. B1f;

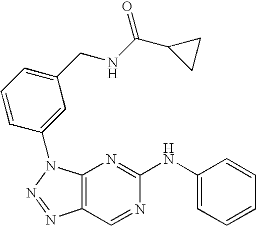

Co. No. 14; Ex. B1g;

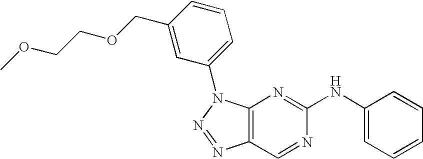

Co. No. 2; Ex. B1b ; mp. 143° C.

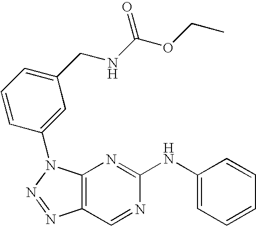

Co. No. 15; Ex. B1g;

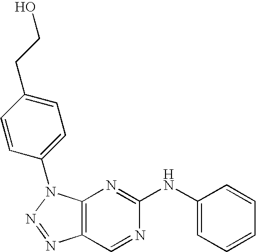

Co. No. 16; Ex. B1b; mp. 175° C.

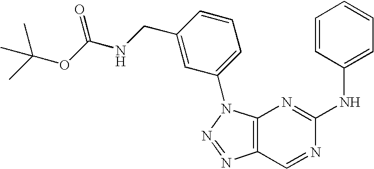

Co. No. 5; Ex. B1e; mp. 196° C.

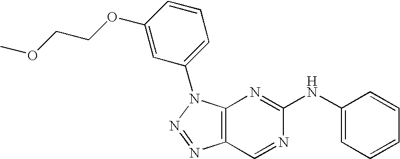

Co. No. 3; Ex. B1c; mp. 164° C.

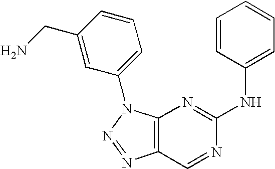

Co. No. 9; Ex. B3; mp. >260° C.
•2HCl

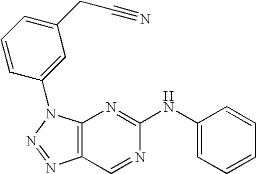

Co. No. 1; Ex. B1a; mp. 218° C.

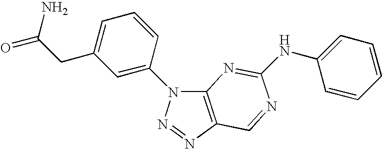

Co. No. 17; Ex. B1b; mp. 224° C.

### C. Analytical Data

The mass of the compounds was recorded with LCMS (liquid chromatography mass spectrometry). The data are gathered in Table 2 below.

### LCMS Conditions

The HPLC gradient was supplied by a Waters 600 system with a column heater set at 45° C. Flow from the column was split to a Waters 996 photodiode array (PDA) detector and a Waters-Micromass LCT mass spectrometer with an electrospray ionization source operated in positive ionization mode. Reversed phase HPLC was carried out on a Xterra MS C18 column (3.5 mm, 4.6×100 mm) with a flow rate of 1.6 ml/minute. Three mobile phases (mobile phase A 95% 25 mM ammoniumacetate+5% acetonitrile; mobile phase B: acetonitrile; mobile phase C: methanol) were employed to run a gradient condition from 100% A to 35% B and 35% C in 3 minutes, to 50% B and 50% C in 3.5 minutes, to 100% B in 0.5 minute, 100% B for 1 minute and re-equilibrate with 100% A for 1.5 minutes. An injection volume of 10 μL was used. Mass spectra were acquired by scanning from 100 to 1200. The capillary needle voltage was 3 kV and the source temperature was maintained at 120° C. Nitrogen was used a the nebulizer gas. Cone voltage was 10 V for positive ionization mode. Data acquisition was performed with a Waters-Micromass MassLynx-Openlynx data system.

TABLE 2
LCMS parent peak and retention time values

| | Retention | |
|---|---|---|
| | time | LCMS |
| Co. No. | (minutes) | [M + H] |
| 15 | 6.52 | 390 |
| 14 | 9.24 | 386 |
| 7 | 9.56 | 402 |
| 6 | 7.61 | 480 |

### D. Pharmacological Example

The pharmacological activity of the present compounds was examined using the following test.

GSK3beta assays were performed at room temperature in a 100 μl reaction volume of 25 mM Tris (pH 7.4) containing 10 mM MgCl₂.6H₂O, 1 mM DTT, 0.1 mg/ml BSA, 5% glycerol and containing 5.7 ng/μl GSK3β, 5 μM biotinylated phosphorylated CREB peptide, 1 μM ATP, 0.85 μCi/ml ATP-P³³ and a suitable amount of a test compound of formula (I). After one hour, the reaction was terminated by adding 70 μl of Stop mix (0.1 mM ATP, 5 mg/ml streptavidin coated PVT SPA bead pH 11.0). The beads to which the phosphorylated CREB peptide is attached were allowed to settle overnight and the radioactivity of the beads was counted in a microtiterplate scintillation counter and compared with the results obtained in a control experiment (without the presence of a test compound) in order to determine the percentage of GSK3β inhibition. The IC₅₀ value, i.e. the concentration (M) of the test compound at which 50% of GSK3β is inhibited, was calculated from the dose response curve obtained by performing the above-described GSK3β assay in the presence of different amounts of the test compound.

The GSK3alpha assay was performed in the same way as described above for the GSK3beta assay except for the concentration of GSK3alpha which is 0.25 ng/μl.

Table 3 lists ranges (namely pIC₅₀>8; pIC₅₀ ranging between 7 and 8; pIC₅₀<7) of pIC₅₀ values (−log IC₅₀ (M)) obtained in the above-described test for the present compounds.

TABLE 3

| | pIC₅₀ | pIC₅₀ |
|---|---|---|
| Co. No. | (GSK3beta) | (GSK3alpha) |
| 5 | >8 | >8 |
| 8 | <7 | 7-8 |
| 10 | >8 | >8 |
| 13 | <8 | 7-8 |
| 11 | >8 | >8 |
| 12 | 8 | >8 |
| 7 | >8 | >8 |
| 4 | 7-8 | 7-8 |
| 15 | >8 | >8 |
| 3 | 7-8 | >8 |
| 9 | 7-8 | >8 |
| 6 | <7 | <7 |
| 17 | >8 | >8 |
| 1 | >8 | >8 |
| 2 | >8 | >8 |
| 16 | 7-8 | 7-8 |
| 14 | 7-8 | <6 |

1. A compound of formula 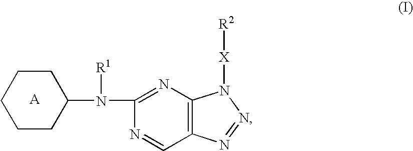
a N-oxide, a pharmaceutically acceptable addition salt, a quaternary amine and a stereochemically isomeric form thereof, wherein
ring A is selected from the group consisting of phenyl, pyridyl, pyrimidinyl, pyridazinyl and pyrazinyl;
R¹ is selected from the group consisting of hydrogen; aryl; formyl; C₁₋₆alkylcarbonyl; C₁₋₆alkyl; C₁₋₆alkyloxycarbonyl; C₁₋₆alkyl substituted with formyl, C₁₋₆alkylcarbonyl, C₁₋₆alkyloxycarbonyl and C₁₋₆alkylcarbonyloxy;
X is selected from the group consisting of a direct bond; —(CH₂)ₙ₃— or —(CH₂)ₙ₄—X₁ₐ—X_{1b}—;
with n₃ representing an integer with value 1, 2, 3 or 4;
with n₄ representing an integer with value 1 or 2;
with X₁ₐ representing O, C(═O) or NR³; and
with X_{1b} representing a direct bond and C₁₋₂alkyl;
R² is selected from the group consisting of C₃₋₇cycloalkyl; phenyl; a 4, 5, 6- or 7-membered monocyclic heterocycle containing at least one heteroatom selected from O, S or N; benzoxazolyl and a radical of formula 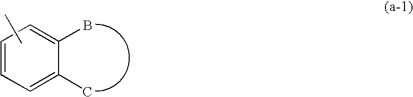
wherein —B—C— is selected from the group consisting of a bivalent radical of formula
—CH₂—CH₂—CH₂— (b-1);
—CH₂—CH₂—CH₂—CH₂— (b-2);
—X₁—CH₂—CH₂—(CH₂)ₙ— (b-3);
—X₁—CH₂—(CH₂)ₙ—X₁— (b-4);
—X₁—(CH₂)_{n′}—CH═CH— (b-5); and
—CH═N—X₁— (b-6);
with X₁ is O or NR³;
n is an integer with value 0, 1, 2 or 3;
n′ is an integer with value 0 or 1;
wherein said R² substituent, where possible, may optionally be substituted with at least one substituent selected from the group consisting of halo; hydroxy; C₁₋₆alkyl optionally substituted with at least one R⁸ substituent; C₂₋₆alkenyl or C₂₋₆alkynyl, each optionally substituted with at least one R⁸ substituent; polyhaloC₁₋₆alkyl optionally substituted with at least one R⁸ substituent; C₁₋₆alkyloxy optionally substituted with at least one R⁸ substituent; polyhaloC₁₋₆alkyloxy optionally substituted with at least one R⁸ substituent; C₁₋₆alkylthio; polyhaloC₁₋₆alkylthio; C₁₋₆alkyloxycarbonyl; C₁₋₆alkylcarbonyloxy; C₁₋₆alkylcarbonyl; polyhaloC₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; —NR³—CN; aryloxy; arylthio; arylcarbonyl; arylC₁₋₄alkyl; arylC₁₋₄alkyloxy; a 5- or 6-membered monocyclic heterocycle containing at least one heteroatom selected from O, S or N and said 5- or 6-membered monocyclic heterocycle optionally being substituted with at least one substituent selected from R⁷; and 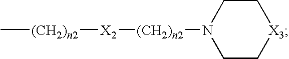
wherein
n2 is selected from the group consisting of an integer with value 0, 1, 2, 3 and 4;
with X₂ is selected from the group consisting of O, NR³ and a direct bond;
with X₃ is selected from the group consisting of O, CH₂, CHOH, CH—N(R³)₂, NR³ and N—C(═O)—C₁₋₄alkyl;
R³ is selected from the group consisting of hydrogen; C₁₋₄alkyl and C₂₋₄alkenyl;
R⁴ and R⁵ each independently is selected from the group consisting of hydrogen; cyano; C₁₋₆alkylcarbonyl optionally substituted with C₁₋₄alkyloxy or carboxyl; C₁₋₆alkyloxy carbonyl; C₃₋₇cycloalkylcarbonyl; adamantanylcarbonyl; C₁₋₄alkyloxyC₁₋₄alkyl; C₁₋₄alkyl substituted with C₁₋₄alkyl-NR³—; C₁₋₆alkyl optionally substituted with at least one substituent selected from halo, hydroxy, cyano, carboxyl, C₁₋₄alkyloxy, polyhalo-C₁₋₄alkyl, C₁₋₄alkyloxyC₁₋₄alkyloxy, NR^{4a}R^{5a}, C(═O)NR^{4a}R^{5a} or 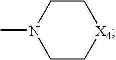
with X₄ representing O, CH₂, CHOH, CH—N(R³)₂, NR³ and N—C(═O)—C₁₋₄alkyl;
R^{4a} and R^{5a} each independently is selected from the group consisting of hydrogen; C₁₋₄alkyl; C₁₋₄alkylcarbonyl and a 5- or 6-membered monocyclic heterocycle containing at least one heteroatom selected from O, S or N;
R⁶ is selected from the group consisting of C₁₋₄alkyl optionally substituted with hydroxy; polyhaloC₁₋₄alkyl and NR⁴R⁵;
R⁷ is selected from the group consisting of halo; hydroxy; C₁₋₆alkyl optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶, C₂₋₆alkenyl or C₂₋₆alkynyl, each optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶; polyhaloC₁₋₆alkyl; C₁₋₆alkyloxy optionally substituted with carboxyl; polyhaloC₁₋₆alkyloxy; C₁₋₆alkylthio; polyhaloC₁₋₆alkylthio; C₁₋₆alkyloxycarbonyl; C₁₋₆alkylcarbonyloxy; C₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; and —NR³—CN;
R⁸ is selected from the group consisting of hydroxy, cyano, carboxyl, C₁₋₄alkyloxy, C₁₋₄alkyloxyC₁₋₄alkyloxy,
C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶;
n1 is an integer with value 1 or 2;
aryl is phenyl or phenyl substituted with at least one substituent selected from the group consisting of halo, C₁₋₆alkyl, C₃₋₇cycloalkyl, C₁₋₆alkyloxy, cyano, nitro, polyhaloC₁₋₆alkyl and polyhaloC₁₋₆alkyloxy, provided that N,3-diphenyl-3H-1,2,3-triazolo[4,5-d]pyrimidin-5-amine is not included.

2. A compound according to claim 1 provided that when ring A is phenyl, X is a direct bond and R² is phenyl, then said R² phenyl must be substituted.

3. A compound according to claim 1 provided that when X is a direct bond and R² is phenyl, then said R² phenyl must be substituted.

4. A compound according to claim 2 wherein ring A is phenyl.

5. A compound according to claim 3 wherein X represents a direct bond.

6. A compound according to claim 2 wherein R¹ represents hydrogen.

7. A compound according to claim 1 wherein
ring A represents phenyl;
R¹ is hydrogen;
X is a direct bond;
R² is phenyl optionally substituted with halo; C₁₋₆alkyl optionally substituted with at least one substituent selected from the group consisting of hydroxy, cyano, carboxyl, C₁₋₄alkyloxy, C₁₋₄alkyloxyC₁₋₄alkyloxy and NR⁴R⁵ wherein R⁴, R⁵ each independently represent hydrogen; C₁₋₆alkylcarbonyl optionally substituted with C₁₋₄alkyloxy; C₁₋₆alkyloxycarbonyl; C₃₋₇cycloalkyl-carbonyl; or adamantanylcarbonyl.

8. (canceled)

9. The use of a compound for the prevention or the treatment of a disease mediated through GSK3 comprising administering to a patient in need of treatment of therapeutically effective amounts of a compound of formula 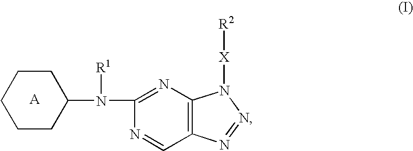
a N-oxide, a pharmaceutically acceptable addition salt, a quaternary amine and a stereochemically isomeric form thereof, wherein
ring A is selected from the group consisting of phenyl, pyridyl, pyrimidinyl, pyridazinyl and pyrazinyl;
R¹ is selected from the group consisting hydrogen; aryl; formyl; C₁₋₆alkylcarbonyl; C₁₋₆alkyl; C₁₋₆alkyloxycarbonyl; C₁₋₆alkyl substituted with formyl, C₁₋₆alkylcarbonyl, C₁₋₆alkyloxycarbonyl and C₁₋₆alkylcarbonyloxy;
X is selected from the group consisting of a direct bond; —(CH₂)ₙ₃—(CH₂)ₙ₄—X₁ₐ—X_{1b}—;
n₃ is selected from the group consisting of an integer with value 1, 2, 3 and 4;
n₄ is selected from the group consisting of an integer with value 1 and 2;
X₁ₐ is selected from the group consisting of O, C(═O) and NR³; and
X_{1b} is selected from the group consisting of a direct bond and C₁₋₂alkyl;
R² is selected from the group consisting of C₃₋₇cycloalkyl; phenyl; a 4, 5, 6- or 7-membered monocyclic heterocycle containing at least one heteroatom selected from O, S or N; benzoxazolyl and a radical of formula 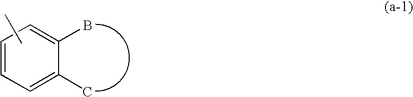
wherein —B—C— is selected from the group consisting of a bivalent radical of formula
—CH₂—CH₂—CH₂— (b-1);
—CH₂—CH₂—CH₂—CH₂— (b-2);
—X₁—CH₂—CH₂—(CH₂)ₙ— (b-3);
—X₁—CH₂—(CH₂)ₙ—X₁— (b-4);
—X₁—(CH₂)_{n′}—CH═CH— (b-5);
—CH═N—X₁— (b-6);
with X₁ is selected from the group consisting of O and NR³;
n is selected from the group consisting of an integer with value 0, 1, 2 and 3; n′ is selected from the group consisting of an integer with value 0 and 1;
wherein said R² substituent, where possible, may optionally be substituted with at least one substituent selected from the group consisting of halo; hydroxy; C₁₋₆alkyl optionally substituted with at least one R⁸ substituent; C₂₋₆alkenyl or C₂₋₆alkynyl, each optionally substituted with at least one R⁸ substituent; polyhaloC₁₋₆alkyl optionally substituted with at least one R⁸ substituent; C₁₋₆alkyloxy optionally substituted with at least one R⁸ substituent; polyhaloC₁₋₆alkyloxy optionally substituted with at least one R⁸ substituent; C₁₋₆alkylthio; polyhaloC₁₋₆alkylthio; C₁₋₆alkyloxycarbonyl; C₁₋₆alkylcarbonyloxy; C₁₋₆alkylcarbonyl; polyhaloC₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; —NR³—CN; aryloxy; arylthio; arylcarbonyl; arylC₁₋₄alkyl; arylC₁₋₄alkyloxy; a 5- or 6-membered monocyclic heterocycle containing at least one heteroatom selected from O, S or N and said 5- or 6-membered monocyclic heterocycle optionally being substituted with at least one substituent selected from R⁷; and 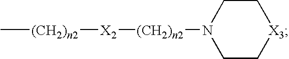
n2 is selected from the group consisting of an integer with value 0, 1, 2, 3 and 4;
X₂ is selected from the group consisting of O, NR³ and a direct bond;
X₃ is selected from the group consisting of O, CH₂, CHOH, CH—N(R³)₂, NR³ or
N—C(═O)—C₁₋₄alkyl;
R³ is selected from the group consisting of hydrogen; C₁₋₄alkyl and C₂₋₄alkenyl;
R⁴ and R⁵ each independently are selected from the group consisting of hydrogen; cyano; C₁₋₆alkylcarbonyl optionally substituted with C₁₋₄alkyloxy and carboxyl; C₁₋₆alkyloxycarbonyl;
C₃₋₇cycloalkylcarbonyl; adamantanylcarbonyl; C₁₋₄alkyloxyC₁₋₄alkyl; C₁₋₄alkyl substituted with C₁₋₄alkyl-NR³—; C₁₋₆alkyl optionally substituted with at least one substituent selected from halo, hydroxy, cyano, carboxyl, C₁₋₄alkyloxy, polyhalo-C₁₋₄alkyl, C₁₋₄alkyloxyC₁₋₄alkyloxy, NR^{4a}R^{5a}, C(═O)NR^{4a}R^{5a} and 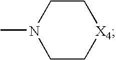
wherein with X₄ representing O, CH₂, CHOH, CH—N(R³)₂, NR³ or N—C(═O)—C₁₋₄alkyl;
R^{4a} and R^{5a} each independently are selected from the group consisting of hydrogen; C₁₋₄alkyl; C₁₋₄alkylcarbonyl and a 5- or 6-membered monocyclic heterocycle containing at least one heteroatom selected from O, S or N;
R⁶ is C₁₋₄alkyl optionally substituted with hydroxy; polyhaloC₁₋₄alkyl or NR⁴R⁵;
R⁷ is selected from the group consisting of halo; hydroxy; C₁₋₆alkyl optionally substituted with at least one substituent selected from the group consisting of hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶, C₂₋₆alkenyl and C₂₋₆alkynyl, each optionally substituted with at least one substituent selected from hydroxy, cyano, carboxyl, C₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ or —NR³—S(═O)ₙ₁—R⁶; polyhaloC₁₋₆alkyl; C₁₋₆alkyloxy optionally substituted with carboxyl; polyhaloC₁₋₆alkyloxy; C₁₋₆alkylthio; polyhaloC₁₋₆alkylthio; C₁₋₆alkyloxycarbonyl; C₁₋₆alkylcarbonyloxy; C₁₋₆alkylcarbonyl; cyano; carboxyl; NR⁴R⁵; C(═O)NR⁴R⁵; —NR³—C(═O)—NR⁴R⁵; —NR³—C(═O)—R³; —S(═O)ₙ₁—R⁶; —NR³—S(═O)ₙ₁—R⁶; —S—CN; and —NR³—CN;
R⁸ is selected from the group consisting of hydroxy, cyano, carboxyl, C₁₋₄alkyloxy, C₁₋₄alkyloxyC₁₋₄alkyloxy, C₁₋₄alkylcarbonyl, C₁₋₄alkyloxycarbonyl, C₁₋₄alkylcarbonyloxy, NR⁴R⁵, —C(═O)—NR⁴R⁵, —NR³—C(═O)—NR⁴R⁵, —S(═O)ₙ₁—R⁶ and —NR³—S(═O)ₙ₁—R⁶;
n1 is an integer with value 1 or 2;
aryl is phenyl or phenyl substituted with at least one substituent selected from the group consisting of halo, C₁₋₆alkyl, C₃₋₇cycloalkyl, C₁₋₆alkyloxy, cyano, nitro, polyhaloC₁₋₆alkyl and polyhaloC₁₋₆alkyloxy.

10. The use of a compound as defined in claim 1 for the prevention or the treatment of bipolar disorder (in particular manic depression), diabetes, Alzheimer's disease, leukopenia, FTDP-17 (Fronto-temporal dementia associated with Parkinson's disease), cortico-basal degeneration, progressive supranuclear palsy, multiple system atrophy, Pick's disease, Niemann Pick's disease type C, Dementia Pugilistica, dementia with tangles only, dementia with tangles and calcification, Downs syndrome, myotonic dystrophy, Parkinsonism-dementia complex of Guam, aids related dementia, Postencephalic Parkinsonism, prion diseases with tangles, subacute sclerosing panencephalitis, frontal lobe degeneration (FLD), argyrophilic grains disease, subacute sclerotizing panencephalitis (SSPE) (late complication of viral infections in the central nervous system), inflammatory diseases, depression, cancer, dermatological disorders, neuroprotection, schizophrenia or pain.

11. The use of a compound as claimed in claim 10 for the prevention or the treatment of Alzheimer's disease; diabetes; cancer; inflammatory diseases; bipolar disorder; depression; pain.

12. A pharmaceutical composition comprising a pharmaceutically acceptable carrier and as active ingredient a therapeutically effective amount of a compound as claimed in claim 1.

13. A process for preparing a pharmaceutical composition as claimed in claim 12 characterized in that a therapeutically effective amount of a compound as claimed in claim 1 is intimately mixed with a pharmaceutically acceptable carrier.

14. A process for preparing a compound as claimed in claim 1, wherein
a) reacting an intermediate of formula (II) with an intermediate of formula (III) in the presence of a suitable solvent, optionally in the presence of a suitable base, 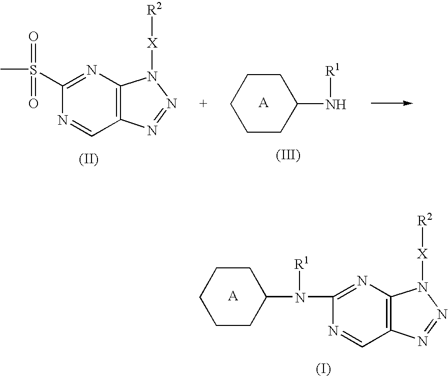
wherein R¹, R², X and ring A are as defined in claim 1;
b) by cyclizing an intermediate of formula (IV) in the presence of a nitrite salt, a suitable acid, and optionally in the presence of a suitable solvent, 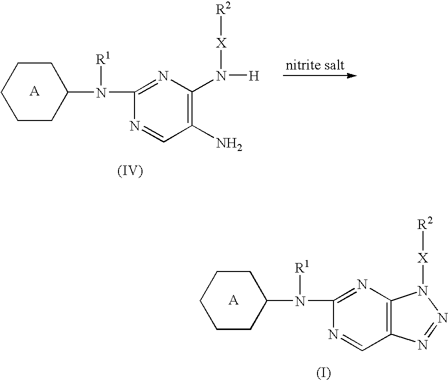
wherein R¹, R², X and ring A are as defined in claim 1;
c) by reacting an intermediate of formula (V) with an intermediate of formula (III) in the presence of a suitable solvent, optionally in the presence of a suitable base, 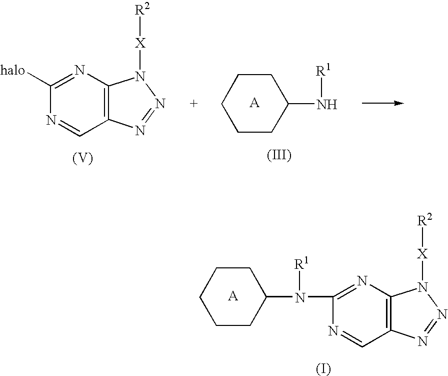
wherein R¹, R², X and ring A are as defined in claim 1;
and optionally thereafter, converting the compounds of formula (I), into a therapeutically active non-toxic acid addition salt by treatment with an acid, or into a therapeutically active non-toxic base addition salt by treatment with a base, or conversely, converting the acid addition salt form into the free base by treatment with alkali, or converting the base addition salt into the free acid by treatment with acid; and, if desired, preparing stereochemically isomeric forms, quaternary amines or N-oxide forms thereof.
